SEQUENCE LISTING (8 sequences)

SEQ ID NO: 1 - protein, 179 aa, Artificial Sequence
  hDectin1 (A) domain
IEGRNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 2 - protein, 15 aa, Artificial Sequence
  Hinge
EPKSCDKTHTCPPCP

SEQ ID NO: 3 - protein, 217 aa, Artificial Sequence
  hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 23 aa, Artificial Sequence
  Secretion signal peptide
MRVPAQLLGLLLLWLSGARCSGS

SEQ ID NO: 5 - protein, 440 aa, Artificial Sequence
  hDectin-1 (A) + Fc(G1)
MRVPAQLLGLLLLWLSGARCSGSHHHHHHIEGRNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 143 aa, Artificial Sequence
  hDectin-1 (B) domain
TPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 7 - protein, 134 aa, Artificial Sequence
  hdectin-1 (C) domain
GVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 8 - protein, 7 aa, Artificial Sequence
  Residues 66-72 of human Dectin-1 receptor polypeptide
TMAIWRS

(19) United States
(12) Patent Application Publication
(10) Pub. No.: US 2023/0257444 A1
(43) Pub. Date: Aug. 17, 2023

(54) FUSION PROTEIN AND COMBINATIONS THEREOF

(71) Applicant: Agency for Science, Technology and Research, Singapore (SG)

(72) Inventors: Linus Wei Jie Lim, Singapore (SG); Yue Wang, Singapore (SG); Say Kong Ng, Singapore (SG); Kong Peng Lam, Singapore (SG)

(21) Appl. No.: 18/014,992

(22) PCT Filed: Jul. 15, 2021
(86) PCT No.: PCT/SG2021/050411

§ 371 Date: Jan. 6, 2023

(30) Foreign Application Priority Data
Jul. 15, 2020 (SG) 10202006746S

(51) Int. Cl. C07K 14/705

(52) U.S. Cl. CPC C07K 14/7056; C07K 2319/30

(57) ABSTRACT

The present invention relates to a soluble dimeric fusion protein comprising a first and second polypeptides, wherein the first and second polypeptides each comprises a Dectin-1 receptor polypeptide fused to a human Fc domain via a dimerization linker. Methods of using the soluble dimeric fusion protein for immunizing a subject against a fungal infection, preventing or treating a fungal infection in a subject and detecting a fungal infection in a subject are also provided. In one embodiment, a chimeric molecule comprising the fusion protein and a payload is provided. In one embodiment, the payload is Amphotericin B.

### FIELD

The present disclosure relates generally to the field of immunology. In particular, the disclosure teaches a soluble dimeric fusion protein comprising a first and second polypeptides, wherein the first and second polypeptides each comprises a Dectin-1 receptor polypeptide fused to a human Fc domain via a dimerization linker.

### BACKGROUND

Invasive mycosis is a potentially fatal opportunistic infection that affects immunocompromised individuals who are suffering from pre-existing medical conditions. Despite advances in healthcare, there is an ever increasing number of patients who are immune-suppressed or immune-deficient for prolonged periods of time, leading to an increased risk of contracting invasive mycosis. This group of patient includes cancer patients, HIV patients, organ or stem cell transplant patients and includes critically ill or elderly patients.

The global burden of invasive mycoses is difficult to quantify accurately because of the lack of comprehensive systemic national and global surveillance programmes and complications of accurately diagnosing the infections. Invasive mycoses are estimated to kill 1.5 million people every year. The vast majority of invasive mycoses related deaths are caused by Candida, Aspergillus, Cryptococcus and Pneumocystis.

The incidence of systemic candidiasis, for example, has increased dramatically over the past 50 years, reflecting increasingly interventional medical care that cause compromised immunity. According to estimates, invasive candidiasis affects between 250,000 to more than 400,000 people worldwide every year and is the cause of more than 50,000 deaths. Incidence rates of candidemia have been reported to be between 2 and 14 cases per 100,000 persons in population-based studies. Candida species are the most common fungal pathogens to cause life threatening invasive mycoses in patients.

Current small molecule drugs while capable of managing a moderate fungal mycoses are ill suited for prophylaxis or long term passive protection during a patient's immunocompromised state.

Accordingly, there is a need to overcome, or at least to alleviate, one or more of the above-mentioned problems.

### SUMMARY

Disclosed herein is a soluble dimeric fusion protein comprising a first and second polypeptides, wherein the first and second polypeptides each comprises a human Dectin-1 receptor polypeptide fused to a human Fc domain via a dimerization linker, wherein the first and second polypeptides form a dimeric fusion protein via association between the dimerization linkers on each of the first and second polypeptides.

Disclosed herein is a chimeric molecule comprising a fusion protein as defined herein, and a heterologous moiety.

Disclosed herein is an isolated polynucleotide comprising a nucleic acid sequence encoding the fusion protein as defined herein.

Disclosed herein is a construct comprising a nucleic acid sequence encoding the fusion protein as defined herein.

Disclosed herein is a host cell containing a construct as defined herein.

Disclosed herein is a method of preparing a fusion protein as defined herein, the method comprising expressing the fusion protein with a host cell as defined herein, and purifying the fusion protein.

Disclosed herein is a pharmaceutical composition comprising a fusion protein as defined herein, and a pharmaceutically acceptable carrier.

Disclosed herein is a fusion protein as defined herein for use as a medicament.

Disclosed herein is a method of immunizing a subject against a fungal infection, the method comprising administering to the subject with a therapeutically effective amount of a fusion protein as defined herein to immunize the subject against fungal infection.

Disclosed herein is a method of preventing or treating a fungal infection in a subject, the method comprising administering to the subject a therapeutically effective amount of a fusion protein as defined herein to prevent or treat the fungal infection in the subject.

Disclosed herein is a kit comprising a fusion protein as defined herein.

Disclosed herein is a method of detecting a fungal infection in a subject, the method comprising the step of determining the level of β-glucan in a sample with a fusion protein as defined herein, wherein an increased level of β-glucan as compared to a reference indicate the presence of a fungal infection in the subject.

### BRIEF DESCRIPTION OF THE DRAWINGS

Some embodiments of the present invention will now be described by way of non-limiting example only, with reference to the accompanying drawings in which:

FIG. 1: Function of Dectin1-Fc

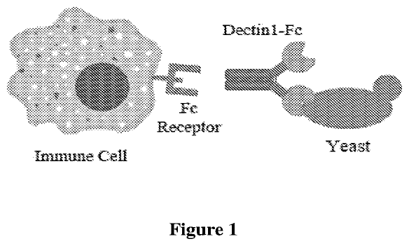

FIG. 2: General process flow for protein construct evaluation of hDectin1 fragments and hDectin1-Fc

FIG. 3: Vector map and western blot hDectin1fragment screening. (A) nHis-hDec1(A) screen. (B) nHis-hDec1(B) screen. (C) nHis-hDec1(C) screen. (D) hDec1(A)-cHis screen. (E) hDec1(B)-cHis screen. (F) hDec1(C)-cHis screen.

FIG. 4: Vector map and western blot of hDectin1-Fc screening (A) Vector map of nHis-hDectin1(A)-FcG1. (B) Vector map of nHis-hDectin1(B)-FcG1. (C) Western blot of surviving minipools with expected mass of 100 kDa. A1-A6, represents minipools transfected with vector A. B, represents minipools transfected with vector B1-B2.

FIG. 5: Preliminary screening of CHO K1 polyclonal pools expressing hDectin1-Fc. (A) Vector map of hDectin1-Fc in CHO K1. (B) Western blot of surviving transfected CHO K1 minipools with expected mass of 100 kDa. (C) Crude titres of A3 and A6 minipools grown in HyClone PF CHO media for 7 days in batch mode.

FIG. 6: Preliminary screening of CHO DG44 polyclonal pools expressing hDectin1-Fc. (A) Vector map of hDectin1-Fc in CHO DG44. (B) Western blot of surviving transfected CHO DG44 in 50 nM MTX. (C) Western blot of surviving transfected CHO DG44 in 150 nM MTX. (D) Western blot of surviving transfected CHO DG44 in 250 nM MTX with expected mass of 100 kDa. (E) Crude titres of F6 and F11 minipools grown in HyClone PF CHO media for 7 days in batch mode.

FIG. 7: Growth profiles comparing CHO K1 and CHO DG44 expressing hDectin1-Fc in various culture conditions. (A) 2 L shake flask culture of CHO DG44 and CHO K1 at 2×10⁵ cells/ml and 5×10⁵ cells/ml seeding density in Hyclone media. (B)) 2 L shake flask culture of CHO DG44 and CHO K1 at 2×10⁵ cells/ml and 5×10⁵ cells/ml seeding density in Excell media. (C) 2 L shake flask culture of CHO DG44 and CHO K1 at 2×10⁵ cells/ml and 5×10⁵ cells/ml seeding density in Actipro media. (D) 5 L bioreactor culture of CHO DG44 and CHO K1 at 3×10⁵ cells/ml seeding density at 37° C. constant temperature culture or initial 37° C. followed by temperature shift to 33° C.

FIG. 8: Titer profile of CHO K1 A6 and CHO DG44 F11 expressing hDectin1-Fc in various culture conditions of seeding density and media. SF-Shake flask culture. BR-bioreactor culture.

FIG. 9: Structure analysis of hDectin1-Fc (A), Western Blot of reduced and non-reduced hDectin1-Fc. (B) Schematic structure of hDectin1-Fc (A).

FIG. 10: Immunofluorescence of hDectin1-Fc binding to Candida albicans.

FIG. 11: Surface plasmon resonance respond-time graphs for hDectin1-Fc and Herceptin against bound Fcγ receptors and FcRn.

FIG. 12: In vitro CFU assay of human primary immune cells against Candida albicans with varying concentrations of hDectin1-Fc. Effector to target ratio of 10⁵ immune cells to 10⁵ Candida albicans and incubated at 37° C. for 1 hour. All experiments were performed three times and data analyzed using one way ANOVA. *, P<0.05; **P<0.01; ***P<0.001.

FIG. 13: In vitro combination therapy dilution assay of Amphotericin B and hDectin1-Fc at fixed concentrations with 10⁵ Candida albicans and 10⁵ human primary macrophages. The assay was incubated for 24 hours at 37° C. before the minimum inhibitory concentration and minimum fungicidal concentration were determined. Minimum Inhibitory Concentration (MIC). Minimum Fungicidal Concentration (MFC).

FIG. 14: Concentration-time plots of hDectin1-Fc serum levels at 4 mg, 2 mg, 1 mg and 0.5 mg. Each plot represents an average of 3 mice. (A) Concentration-time plot over 20 days. (B) Concentration-time plot in the first 48 hours.

FIG. 15: Effect of passive immunization with hDectin1-Fc in mice challenged with Candida albicans. Eight mice per group were treated intraperitoneally with a bolus dose of PBS alone or hDectin1-Fc at various doses. Two hours later, the mice were challenged with SC5314 Candida albicans at the respective inoculum. (A) 0.5 million inoculum, (B) 0.25 million inoculum, (C) 0.1 million inoculum and (D) 0.05 million inoculum. Kaplan-Meier Survival plots were compared for statistical significance using the Mantel-Cox log rank test. *, P≤0.05; **; P≤0.01; ***; P≤0.001.

FIG. 16: Therapy comparison with hDectin1-Fc, Amphotericin B (AmB) and Combination therapy of Amphotericin B and hDectin1-Fc in mice challenged with Candida albicans. Eight mice per group were treated intraperitoneally with a bolus dose of 1 mg hDectin1-Fc, 0.05 mg/kg/day AmB once daily for 7 days, or a combination therapy of 1 mg hDectin1-Fc and 0.05 mg/kg/day AmB once daily for 7 days. Two hours post hDectin1-Fc treatment, the mice were challenged with 0.5 million SC5314 Candida albicans at the respective inoculum. (A) Survival plot post 7 days, (B) Survival plot post 2 weeks, (C) Survival plot post 1 month. Kaplan-Meier Survival plots were compared for statistical significance using the Mantel-Cox log rank test. *, P≤0.05; **; P≤0.01; ***; P≤0.001.

FIG. 17: (A) hDectin1-AmB construct schematic. (B) hDectin1-Fc-AmB construct schematic.

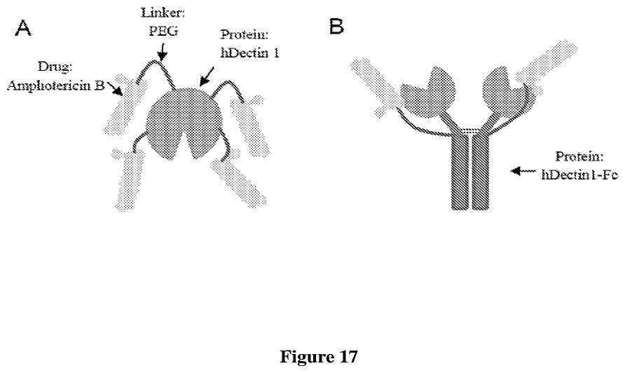

FIG. 18: Production process of hDectin1-AmB

FIG. 19: Screening of hDectin1 CHO cell production vehicle (A) Vector map and western blot of nHis-hDec1(A) in CHO K1 cells screen. (B) Vector map and western blot of nHis-hDec1(A) in CHO DG44 cells screen.

FIG. 20: Synthesis of PEG methyl terminated linker with Amphotericin B (A) Synthetic route of Polyethylene glycol conjugation with amino functional group of Amphotericin B. (B) Synthetic route of Polyethylene glycol conjugation with carboxylic functional group of Amphotericin B.

FIG. 21: Schematic synthesis of hDectin1 & PEGylated Amphotericin B.

FIG. 22: Synthesis of Thiol labile maleimide terminated C-linked PEG.

### DETAILED DESCRIPTION

Disclosed herein is soluble dimeric fusion protein comprising a first and second polypeptides, wherein the first and second polypeptides each comprises a Dectin-1 receptor polypeptide fused to a human Fc domain via a dimerization linker, wherein the first and second polypeptides form a dimeric fusion protein via association between the dimerization linkers on each of the first and second polypeptides.

The Dectin-1 receptor polypeptide may be a human Dectin-1 receptor polypeptide. The human Dectin-1 receptor polypeptide may comprise or consist an amino acid sequence having at least 70% sequence identity to amino acid 73-247 of human Dectin-1 receptor polypeptide (i.e. SEQ ID NO: 1). Without being bound by theory, amino acid 73-247 of human Dectin-1 receptor polypeptide is found to be an ideal length with good expression and solubility. Amino acid 73-247 of human Dectin-1 receptor polypeptide does not contain the aromatic or hydrophobic residues of MAIW in amino acid 66-72 (i.e. TMAIWRS (SEQ ID NO: 8)) of human Dectin-1 receptor polypeptide and may advantageously help with the expression and stability of the protein. The absence of the aromatic or hydrophobic residues may also help to avoid interactions at the site of fusion with IgG1 Fc.

The human Dectin receptor polypeptide may comprise or consists of amino acid residues 73-247 of the human Dectin-1 receptor polypeptide. In one embodiment, the human Dectin-1 receptor polypeptide comprises or consists an amino acid sequence having at least 70% (or at least 80%, 85%, 90% or 95%) sequence identity to an amino acid sequence of SEQ ID NO: 1.

In one embodiment, the human Dectin receptor polypeptide does not contain amino acid residues 66-72 (i.e. TMAIWRS (SEQ ID NO: 8)) of the human Dectin-1 receptor polypeptide.

The terms “polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

By “recombinant polypeptide” is meant a polypeptide made using recombinant techniques, i.e., through the expression of a recombinant polynucleotide.

In one embodiment, the soluble dimeric fusion protein is a recombinant soluble dimeric fusion protein.

TABLE 1

| | | SEQ |
|---|---|---|
| | | ID |
| Name | Sequence | NO: |
| hDectin1 | IEGRNSGSNT LENGYFLSRN KENHSQPTQS | 1 |
| (A) | SLEDSVTPTK AVKTTGVLSS PCPPNWIIYE | |
| domain | KSCYLFSMSL NSWDGSKRQC WQLGSNLLKI | |
| | DSSNELGFIV KQVSSQPDNS FWIGLSRPQT | |
| | EVPWLWEDGS TFSSNLFQIR TTATQENPSP | |
| | NCVWIHVSVI YDQLCSVPSY SICEKKFSM | |
| Hinge | EPKSCDKTHT CPPCP | 2 |
| hIgG1 Fc | APELLGGPSV FLFPPKPKDT LMISRTPEVT | 3 |
| | CVVVDVSHED PEVKFNWYVD GVEVHNAKTK | |
| | PREEQYNSTY RVVSVLTVLH QDWLNGKEYK | |
| | CKVSNKALPA PIEKTISKAK GQPREPQVYT | |
| | LPPSRDELTK NQVSLTCLVK GFYPSDIAVE | |
| | WESNGQPENN YKTTPPVLDS DGSFFLYSKL | |
| | TVDKSRWQQG NVFSCSVMHE ALHNHYTQKS | |
| | LSLSPGK | |
| Secretion | MRVPAQLLGL LLLWLSGARC SGS | 4 |
| signal | | |
| peptide | | |
| hDectin-1 | MRVPAQLLGL LLLWLSGARC SGSHHHHHHI | 5 |
| (A) + | EGRNSGSNTL ENGYFLSRNK ENHSQPTQSS | |
| Fc(G1) | LEDSVTPTKA VKTTGVLSSP CPPNWIIYEK | |
| | SCYLFSMSLN SWDGSKRQCW QLGSNLLKID | |
| | SSNELGFIVK QVSSQPDNSF WIGLSRPQTE | |
| | VPWLWEDGST FSSNLFQIRT TATQENPSPN | |
| | CVWIHVSVIY DQLCSVPSYS ICEKKFSMEP | |
| | KSCDKTHTCP PCPAPELLGG PSVFLFPPKP | |
| | KDTLMISRTP EVTCVVVDVS HEDPEVKFNW | |
| | YVDGVEVHNA KTKPREEQYN STYRVVSVLT | |
| | VLHQDWLNGK EYKCKVSNKA LPAPIEKTIS | |
| | KAKGQPREPQ VYTLPPSRDE LTKNQVSLTC | |
| | LVKGFYPSDI AVEWESNGOP ENNYKTTPPV | |
| | LDSDGSFFLY SKLTVDKSRW QQGNVFSCSV | |
| | MHEALHNHYT QKSLSLSPGK | |
| hDectin-1 | TPTKAVKTTG VLSSPCPPNW IIYEKSCYLF | 6 |
| (B) | SMSLNSWDGS KRQCWQLGSN LLKIDSSNEL | |
| domain | GFIVKQVSSQ PDNSFWIGLS RPQTEVPWLW | |
| | EDGSTFSSNL FQIRTTATQE NPSPNCVWIH | |
| | VSVIYDQLCS VPSYSICEKK FSM | |
| hDectin-1 | GVLSSPCPPN WIIYEKSCYL FSMSLNSWDG | 7 |
| (C) | SKRQCWQLGS NLLKIDSSNE LGFIVKQVSS | |
| domain | QPDNSFWIGL SRPQTEVPWL WEDGSTFSSN | |
| | LFQIRTTATQ ENPSPNCVWI HVSVIYDQLC | |
| | SVPSYSICEK KFSM | |

The dimerization linker may comprise or consist of an amino acid sequence having at least one cysteine residues (e.g. one, two, three or more). The dimerization linker may be a hinge domain of an antibody. In one embodiment, the dimerization linker comprises or consists an amino acid sequence having at least 70% (or at least 80%, 85%, 90% or 95%) sequence identity to an amino acid sequence of SEQ ID NO: 2.

In one embodiment, the first and second polypeptides each comprises a Dectin-1 receptor polypeptide positioned upstream of a dimerization linker, which is in turn positioned upstream of the human Fc domain.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G and I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

As used herein, “Fc portion” encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

The fusion proteins disclosed herein comprise an Fc portion that includes at least a portion of the carboxy-terminus of an immunoglobulin heavy chain. For example, the Fc portion may comprise: a CH2 domain, a CH3 domain, a CH4 domain, a CH2-CH3 domain, a CH2-CH4 domain, a CH2-CH3-CH4 domain, a hinge-CH2 domain, a hinge-CH2-CH3 domain, a hing-CH2-CH4 domain, or a hinge-CH2-CH3-CH4 domain. The Fc domain may be derived from antibodies belonging any of the immunoglobulin classes, i.e., IgA, IgD, IgE, IgG, or IgM or any of the IgG antibody subclasses, i.e., IgG1, IgG2, IgG3, and IgG4. The Fc domain may be a naturally occurring Fc sequence, including natural allelic or splice variants. Alternatively, the Fc domain may be a hybrid domain comprising a portion of an Fc domain from two or more different Ig isotypes, for example, an IgG2/IgG4 hybrid Fc domain. In one embodiment, the Fc domain is derived from a human immunoglobulin molecule.

In one embodiment, the Fc domain is an IgG1 Fc domain. In one embodiment, the IgG1 Fc domain comprises or consists of an amino acid sequence having at least 70% (or at least 80%, 85%, 90% or 95%) sequence identity to an amino acid sequence of SEQ ID NO: 3.

In one embodiment, the fusion protein comprises or consists of an amino acid sequence having at least 70% (or at least 80%, 85%, 90%, or 95%) sequence identity to SEQ ID NO: 5.

In one embodiment, the fusion protein specifically binds to β-glucan. The β-glucan may be a β-1,3-glucan from a Candida pathogen.

Disclosed herein is a chimeric molecule comprising a fusion protein as defined herein, and a heterologous moiety.

As used herein, a “chimeric” molecule is one which comprises one or more unrelated types of components or contain two or more chemically distinct regions which can be conjugated to each other, fused, linked, translated, attached via a linker, chemically synthesized, expressed from a nucleic acid sequence, etc. For example, a peptide and a nucleic acid sequence, a peptide and a detectable label, unrelated peptide sequences, and the like. In embodiments in which the chimeric molecule comprises amino acid sequences of different origin, the chimeric molecule includes (1) polypeptide sequences that are not found together in nature (i.e., at least one of the amino acid sequences is heterologous with respect to at least one of its other amino acid sequences), or (2) amino acid sequences that are not naturally adjoined.

In one embodiment, the heterologous moiety comprises a payload.

The term “payload” as used herein refers to any agent that can be conjugated to the fusion protein or chimeric molecule of the present disclosure. The payload can be, for example, an anti-fungal agent, a label, a dye, a polymer, a cytotoxic compound, a radionuclide, an affinity label.

In one embodiment, the payload is an anti-fungal agent. In one embodiment, the anti-fungal agent is Amphotericin B.

The anti-fungal agent may be conjugated to the fusion protein using chemical conjugation techniques that are well known in the art. For example, the anti-fungal agent may be conjugated to the fusion protein via a PEG linker.

In another embodiment, there is provided a Dectin-1 receptor polypeptide conjugated to Amphotericin B.

Disclosed herein is an isolated polynucleotide comprising a nucleic acid sequence encoding the fusion protein as defined herein.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

In one embodiment, there is provided a vector that comprises a nucleic acid encoding the fusion protein as defined herein.

By “vector” is meant a nucleic acid molecule, preferably a DNA molecule derived, for example, from a plasmid, bacteriophage, or virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a construct comprising a nucleic acid sequence encoding the fusion protein as defined herein.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include a RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

Disclosed herein is a host cell containing a construct as defined herein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

In one embodiment, the host cell is a CHO cell (e.g. CHO K1 or CHO DG44).

Disclosed herein is a method of preparing a fusion protein as defined herein, the method comprising expressing the fusion protein with a host cell as defined herein, and purifying the fusion protein.

Disclosed herein is a pharmaceutical composition comprising a fusion protein as defined herein, and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

Pharmaceutical compositions of the present disclosure may be in a form suitable for administration by injection, in a formulation suitable for oral ingestion (such as, for example, capsules, tablets, caplets, elixirs), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. A fusion protein of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the fusion protein can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the fusion protein can be determined by a skilled artisan. In certain embodiments, the fusion protein is administered by injection (e.g., subcutaneously or intravenously) at a dose of about 0.01 to 50 mg/kg, e.g., 0.01 to 0.1 mg/kg, e.g., about 0.1 to 1 mg/kg, about 1 to 5 mg/kg, about 5 to 25 mg/kg, about 10 to 50 mg/kg. The dosing schedule can vary from e.g., once a week to once every 2, 3, or 4 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, there is provided a pharmaceutical composition comprising a fusion protein as defined herein, an anti-fungal agent and a pharmaceutical acceptable carrier.

In one embodiment, there is provided a pharmaceutical combination comprising a fusion protein as defined herein, an anti-fungal agent and optionally a pharmaceutical acceptable carrier.

The pharmaceutical combination may be formulated for sequential or concurrent administration to the subject.

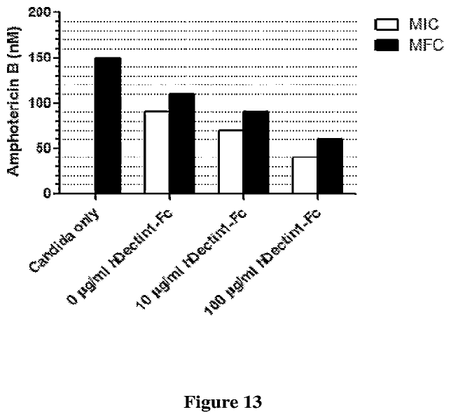

Without being bound by theory, the inventors have shown that a combination of a fusion protein as defined herein and an anti-fungal agent can have synergistic activity (see, for example, FIG. 13).

The terms “a combination” or “in combination with,” it is not intended to imply that the therapeutic agents (i.e. the fusion protein and the anti-fungal agent) must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

Disclosed herein is a fusion protein as defined herein for use as a medicament.

Disclosed herein is a pharmaceutical composition or a pharmaceutical combination as defined herein for use as a medicament.

Disclosed herein is a method of immunizing a subject against a fungal infection, the method comprising administering to the subject with a therapeutically effective amount of a fusion protein as defined herein to immunize the subject against fungal infection.

The terms “subject”, “patient”, “host” or “individual” used interchangeably herein, refer to any subject. The term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

In one embodiment, there is provided a fusion protein as defined herein for use in immunizing the subject against fungal infection.

In one embodiment, there is provided the use of a fusion protein as defined herein in the manufacture of a medicament for immunizing the subject against fungal infection.

In one embodiment, there is provided a method of immunizing a subject against a fungal infection, the method comprising administering to the subject with a therapeutically effective amount of a fusion protein as defined herein and an anti-fungal agent to immunize the subject against fungal infection.

In one embodiment, the fusion protein is bound to a β-glucan molecule.

In one embodiment, there is provided a fusion protein as defined herein and an anti-fungal agent for use in immunizing a subject against fungal infection.

In one embodiment, there is provided the use of a fusion protein as defined herein and an anti-fungal agent in the manufacture of a medicament for immunizing a subject against fungal infection.

In one embodiment, there is provided a method of stimulating an immune response in a subject, the method comprising administering to the subject with a therapeutically effective amount of a fusion protein as defined herein to stimulate an immune response in the subject.

In one embodiment, the immune response is an innate immune response.

In one embodiment, there is provided a fusion protein as defined herein for us in stimulating an immune response in a subject.

In one embodiment, there is provided the use of a fusion protein as defined herein in the manufacture of a medicament for stimulating an immune response in a subject.

The fusion protein may be used to prevent or treat a fungal infection in a subject.

By “fungal infection” is meant the invasion of a host by pathogenic fungi. For example, the infection may include the excessive growth of fungi that are normally present in or on the body of a subject or growth of fungi that are not normally present in or on a subject. More generally, a fungal infection can be any situation in which the presence of a fungal population(s) is damaging to a host body. Thus, a subject is “suffering” from a fungal infection when an excessive amount of a fungal population is present in or on the subject's body, or when the presence of a fungal population(s) is damaging the cells or other tissue of the subject.

The fungal infection being treated can be an infection selected from systemic candidosis, aspergillosis, paracoccidioidomycosis, blastomycosis, histoplasmosis, coccidioidomycosis, sporotrichosis. In certain embodiments, the infection being treated is an infection by Candida albicans, C. parapsilosis, C. glabrata, C. guilliermondii, C. krusei, C. lusitaniae, C. tropicalis, Aspergillus fumigatus, A. flavus, A. terreus. A. niger, A. candidus, A. clavatus, A. ochraceus, Cryptococcus neoformans, Cryptococcus gatti and Pneumocystis jirovecii.

As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In one embodiment, the method further comprises administering a therapeutically effective amount of an anti-fungal agent to the subject.

The anti-fungal agent may be small molecule drugs such as Caspofungin, Fluconazole or Amphotericin B.

In one embodiment, the anti-fungal agent is Amphotericin B. Advantageously, the combination of the fusion protein and Amphotericin B allows the dosage of Amphotericin B to be reduced, leading to enhanced efficacy and lower toxicity (in particular nephrotoxicity).

Amphotericin B may, for example, be Amphotericin B deoxycholate, which can be formulated for intravenous administration to the subject. Alternatively, Amphotericin B may be prepared as a liposomal formulation (e.g. AmBisome) or a lipid complex preparation (e.g. Abelcet) for injection to the subject. Amphotericin B may also be given as an oral preparation (e.g. AmbiOnp).

In one embodiment, Amphotericin B is administered at a dose of about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5 or 5.0 mg/kg/day. In one embodiment, amphotericin B is administered at a dose of about 0.25 mg/kg/day.

In one embodiment, the fusion protein is administered at a dose of about 10, 20, 30, 40, 50, 60, 70, 80, 90 or 100 mg/kg/week. In one embodiment, the fusion protein is administered at a dose of about 50 mg/kg/week.

Disclosed herein is a method of preventing or treating a fungal infection in a subject, the method comprising administering to the subject a therapeutically effective amount of a fusion protein as defined herein to prevent or treat the fungal infection in the subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

In one embodiment, the method further comprises administering a therapeutically effective amount of an anti-fungal agent to the subject.

In one embodiment, the anti-fungal agent is Amphotericin B.

The anti-fungal angent may be administered sequentially or concurrently to the subject.

In one embodiment, there is provided a fusion protein as defined herein for use in preventing or treating a fungal infection in a subject.

In one embodiment, there is provided the use of a fusion protein as defined herein in the manufacture of a medicament for preventing or treating a fungal infection in a subject.

In one embodiment is a method of preventing or treating a fungal infection in a subject, the method comprising administering to the subject a therapeutically effective amount of a fusion protein as defined herein and an anti-fungal agent to prevent or treat the fungal infection in the subject.

In one embodiment, there is provided a fusion protein as defined herein and an anti-fungal agent for use in preventing or treating a fungal infection in a subject.

In one embodiment, there is provided the use of a fusion protein as defined herein and an anti-fungal agent in the manufacture of a medicament for preventing or treating a fungal infection in a subject.

Disclosed herein is a kit comprising a fusion protein as defined herein. The kit may optionally comprise instructions for detecting β-glucan in a sample and/or treating yeast infection in a subject. The kits may also include suitable storage containers (e.g., ampules, vials, tubes, etc.), for each active agent and other included reagents (e.g., buffers, balanced salt solutions, labeling reagents, etc.) for use in administering the active agents to the subject. The active agents and other reagents may be present in the kits in any convenient form, such as, e.g., in a solution or in a powder form. The kits may further include a packaging container, optionally having one or more partitions for housing the active agents and other optional reagents.

Disclosed herein is a method of detecting a fungal infection in a subject, the method comprising the step of determining the level of β-glucan in a sample with a fusion protein of as defined herein, wherein an increased level of β-glucan as compared to a reference indicate the presence of a fungal infection in the subject.

In one embodiment, there is provided a method of treating a fungal infection in a subject, the method comprising a) the step of determining the level of β-glucan in a sample with a fusion protein of as defined herein, wherein an increased level of β-glucan as compared to a reference indicate the presence of a fungal infection in the subject; and b) treating the subject of the fungal infection. The subject may be treated with a therapeutically effective amount of a fusion protein as defined herein or an anti-fungal agent or combination of both.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Certain embodiments of the invention will now be described with reference to the following examples which are intended for the purpose of illustration only and are not intended to limit the scope of the generality hereinbefore described.

### EXAMPLES

Recombinant Plasmid Cloning

The pUC57 plasmids containing genetic sequence of nHis-hDectin1(A), nHis-hDectin1(B), nHis-hDectin1(C) and cHis-hDectin1(A), cHis-hDectin1(B), cHis-hDectin1(C) were bought (Genscript, Nanjing, China). These plasmids were transformed respectively into One Shot™ TOP10 Chemically Competent E. coli (Invitrogen™, Waltham, Mass. USA) according to the manufacturer's protocol and propagated overnight at 37° C. The plasmids were extracted and purified the following day using NucleoBond® Xtra Midi kit (Macherey-Nagel, Duren, Germany). The gene of interest of each plasmid was cut from the respective pUC57 plasmid with restriction enzymes NheI and EcoRI (New England Biolabs, Ipswich, Mass. USA) and the digested mixture ran on a electrophoresis gel. The band containing the gene of interest was excised and purified using NucleoSpin® Gel and PCR Clean-up kit (Macherey-Nagel, Duren, Germany). The gene of interest was then ligated with T4 DNA Ligase (New England Biolabs, Ipswich, Mass. USA) with an in-house vector backbone containing the Zeocin resistance gene or DHFR enzyme selection marker gene. The newly ligated plasmids were then transformed into One Shot™ TOP10 Chemically Competent E. coli (Invitrogen™, Waltham, Mass. USA), propagated overnight and extracted similarly. The purified plasmids were then linearized with BstBI (New England Biolabs, Ipswich, Mass. USA) and ethanol precipitated in preparation for transfection into Chinese Hamster Ovary K1 or DG44 cells. The plasmids containing nHis-hDectin1(A)-Fc and nHis-hDectin1(B)-Fc were cloned in the same process as mentioned above.

Vector Screening & Cell Line Development

The zeocin resistant gene plasmids of nHis-hDectin1(A), nHis-hDectin1(B), nHis-hDectin1(C) and cHis-hDectin1(A), cHis-hDectin1(B), cHis-hDectin1(C) prepared previously were transfected into CHO K1 cells using the Amaxa SG Cell Line 4D-Nucleofector™ X kit with the 4D-Nucleofector™ System (Lonza, Basel, Switzerland) at 10⁷ cells/ml and 4 g of each plasmid respectively. Cells in HyClone PF CHO media (GE Healthcare, Chicago, Ill. USA) were placed in static culture in a 37° C. incubator with a 5% CO₂ atmosphere for 48 hours before being transferred to a 96 well plate at 10⁴ cells/well in HyClone PF CHO media with 600 g/ml Zeocin (Gibco, Carlsbad, Calif. USA). The cells were regularly observed under a Nikon Eclipse Ti-E inverted microscope to identify surviving cell pools and to select confluent wells. Media from confluent wells were collected and used for subsequent Western blot analysis to determine the best expressed hDectin1 fragments to be used for designing hDectin1-Fc. The zeocin resistant gene plasmids containing nHis-hDectin1(A)-Fc and nHis-hDectin1(B)-Fc were transfected in the same process as mentioned above into CHO K1 cells to determine whether nHis-hDectin1(A)-Fc or nHis-hDectin1(B)-Fc is best expressed. nHis-hDectin1(A)-Fc expressing CHO K1 cells were scaled up by passaging into 24 well plates and 6 well plates before transferring into shake flask culture. DHFR gene plasmid of nHis-hDectin1(A)-Fc was similarly transfected into CHO DG44 cells in the process mention above. Cells after transfection were transferred to a 96 well plate at 10⁴ cells/well in HyClone PF CHO media without Hypoxantine, Thymidine and Glycine (−)HT. The transfected CHO DG44 cells that survive the (−)HT were transferred to shake flask culture and subjected at stepwise increasing concentrations of Methotrexate (Merck Sigma Aldrich, Darmstadt, Germany) of 50, 150 and 250 nM. At each concentration of methotrexate, the cells were cultured till their viability improves back to 95%.

Production Cell Vehicle Comparison

Selected polyclonal CHO K1 or DG44 cell pools expressing nHis-hDectin1(A)-Fc were seeded at 5×10⁵ cells/ml in HyClone PF CHO media (GE Healthcare, Chicago, Ill. USA) media with 600 g/ml Zeocin (Gibco, Carlsbad, Calif. USA) or HyClone PF CHO media with 250 nM methotrexate (Merck Sigma Aldrich, Darmstadt, Germany) respectively. The cells were cultured in 250 ml shake flasks (Corning®, Oneonta, N.Y. USA) in a Kuhner Climo-Shaker ISF1-W Incubator at 37° C., 8% CO₂ atmosphere and orbital shaking of 120 rpm for 7 days in a batch culture run. Culture media were collected and the crude titres were compared using Human IgG ELISA Antibody Pair Kit and developed with pNPP ELISA Substrate (STEMCELL Technologies, Vancouver, Canada) on a 96 well plate. The plates were analysed on a Tecan Infinite M200PRO plate reader.

Mammalian Shake Flask Cell Culture for Media Screening

Selected polyclonal CHO K1 or DG44 cell pools expressing hDectin1-Fc in HyClone PF CHO media (GE Healthcare, Chicago, Ill. USA) media with 600 g/ml Zeocin (Gibco, Carlsbad, Calif. USA) and HyClone PF CHO media with 250 nM methotrexate (Merck Sigma Aldrich, Darmstadt, Germany) respectively were cultured in shake flasks in a Kuhner Climo-Shaker ISF1-W Incubator at 37° C., 8% CO₂ atmosphere and orbital shaking of 120 rpm. The cells were seeded at 2×10⁵ cells/ml or 5×10⁵ cells/ml in 2 L shake flasks (Corning®, Oneonta, N.Y. USA) containing either HyClone PF CHO (GE Healthcare, Chicago, Ill. USA), EX-CELL® Advanced CHO Fed-batch (SAFC, Saint Louis Mo. USA), or ActiPro (GE Healthcare, Chicago, Ill. USA) media. The cells were cultured in fed-batch mode as per manufacturer's protocol for each media. Daily cell density was monitored using Beckman Coulter Vi-cell XR cell viability analyser and media profiled using Nova Biomedical Nova BioProfile 400 Biochemistry Analyzer. The cultures were terminated when cell viability dropped to 70-80% viability. Culture medium were collected at the end and processed by centrifugation and 0.22 m sterile filtration to remove cells. The respective culture mediums were then purified via protein A and size exclusion chromatography on a Akta Explorer FPLC (GE Healthcare, Chicago, Ill. USA) and the purified titers determined by Bicinchoninic acid (BCA) protein assay (Thermo Scientific, Rockford, Ill. USA).

Mammalian Bioreactor Cell Culture for Production

hDectin1-Fc expressing CHO K1 and CHO DG44 cells were cultured in 2 L shake flasks (Corning®, Oneonta, N.Y. USA) with EX-CELL® Advanced CHO Fed-batch media (SAFC, Saint Louis Mo. USA) respectively in advance to provide the seed culture. The cells were transferred sterile into a 5 L bioreactor system of Braun Biotech International Biostat-B and basal media added such that volume at the start of culture is 3 L and cell density at 3×10⁵ cells/ml in SAFC EX-CELL® Advanced CHO Fed-batch (SAFC, Saint Louis Mo. USA) basal media. The bioreactor system was aerated via membrane basket with dissolved oxygen (dO2) setpoint at 50%, pH setpoint at 7.0 and agitation at 180 rpm. Feeding of SAFC EX-CELL® Advanced CHO Feed 1 commenced on day 3 and every alternate day thereafter at 10% of culture volume. Daily cell density was monitored using Beckman Coulter Vi-cell XY cell viability counter and media profiled using Nova Biomedical Nova BioProfile 400 Biochemistry Analyzer. The cultures were terminated when cell viability dropped to 70-80%. Culture medium were collected at the end and processed by centrifugation and sterile filtration to remove cells. The respective culture mediums were then purified via protein A and size exclusion chromatography on a Akta Explorer FPLC (GE Healthcare, Chicago, Ill. USA) and the purified titers determined by Bicinchoninic acid (BCA) protein assay (Thermo Scientific, Rockford, 1 L USA).

Western Blot Analysis

Samples from the cell cultures containing either nHis-hDectin1(A), nHis-hDectin1(B), nHis-hDectin1(C), cHis-hDectin1(A), cHis-hDectin1(B), cHis-hDectin1(C), nHis-hDectin1(A)-Fc or nHis-hDectin1(B)-Fc were prepared according to manufacturer's instructions for reduced and non-reduced denaturing conditions and ran on NuPAGE 4-12% Bis-Tris SDS-PAGE Gels (Invitrogen, Carlsbad, Calif. USA) at 200V and 35 mins in MOPS buffer. The Precision Plus Protein™ Dual Colour Standards (BIO-RAD, Singapore) was used as a protein reference standard. The resolved gel was transferred to the PVDF membrane Invitrogen™ iBlot™ 2 Transfer Stacks (Invitrogen, Carlsbad, Calif. USA) using the Invitrogen™ iBlot™ 7-minute dry transfer machine. The PVDF membrane was blocked with 5% Blotting-Grade Blocker Non-fat dry milk (BIO-RAD, Singapore) in TBST buffer and washed trice with TBST buffer after 3 hours. The protein of interest was detected with monoclonal anti-human Dectin1/CLEC7A primary antibody (R&D Systems, Minneapolis, Minn.) and in turn detected with a secondary polyclonal anti mouse HRP conjugate antibody (Promega, Madison, Wis. USA). Each antibody was incubated for 2 hours and washed twice with TBST buffer. TMB (3,3′, 5,5′-tetramethylbenzidine) substrate (Promega, Madison, Wis. USA) was used to achieve chemiluminescence and the blot image captured in a GE Healthcare ImageQuant LAS500.

Immunofluorescence Binding

Candida albicans SC5314 were cultivated overnight on YPD agar (1% Bacto yeast extract, 2% Bacto peptone, 2% D-glucose, and 2% agarose), at 37° C. to obtain unicellular yeast. Additionally, C. albicans was also cultivated in RPMI 1640 (Gibco, Carlsbad, Calif. USA) with 10% FBS to promote filamentous hyphal growth. The fungus cells were dispersed and washed in PBS before resuspension in blocking buffer (PBS+3% BSA) and incubated at room temperature with nHis-hDectin1(A)-Fc for 30 minutes. The cells were washed with blocking buffer and subsequently incubated with with AlexaFluor647-conjugated goat anti-human IgG antibody (Life Technologies, Eugene, Oreg. USA). The cells were washed twice again with blocking buffer to remove the Alexafluor 647 antibody before being fixed on microscope glass slides with 4% Paraformaldehyde (BDH Lab Supplies, England). The fluorescent labelled Candida albicans yeast and hyphae were visualised on a Nikon Eclipse Ti-E inverted microscope.

Surface Plasmon Resonance Analysis

A BIAcore T200 SPR Biosensors (GE Healthcare) was used to assay the interaction of soluble ectodomains of FcR from R&D Systems with hDectin1-Fc and IgG1. Amine coupling via N-hydroxysuccinimide ester was formed on a CM5 sensor chip surface according to a procedure recommended by the manufacturer. Ectodomains were immobilized at acidic pH, resulting in the following densities: FcγRI (#1257-FC-050): 1919 RU, FcγRIIa (#1330-CD-050/CF):1766 RU, FcγRIIb/c (#1875-CD-050): 1972 RU, FcγRIIIa (#4325-FC-050): 2275 RU, FcγRIIIb (#1597-FC-050/CF): 2393 RU, FcRn (#8639-FC-050): 2836. A range of hDectin1-Fc/IgG1 concentrations was injected into flow cells at a flow rate of 20 L/min, with a contact and dissociation time of 300 and 900 seconds, respectively. After each assay cycle, the sensor chip surface was regenerated using 10 mM NaOH. Binding response was recorded as resonance units (RU; 1 RU=1 pg/mm²) continuously, with background binding automatically subtracted. Due to the polyclonal nature of the hDectin1-Fc/IgG1 recombinant proteins used, kinetic constants (kon, koff, t1/2) were not determined, and the KD was calculated by analyzing the concentration-dependence of the steady-state signal reached at the end of the injection using BIA evaluation version 3 software (GE Healthcare) and Scrubber version 2 software (BioLogic Software, Campbell, Australia). The steady-state response was plotted against the concentration of hDectin1-Fc/IgG1 and fitted using Origin software.

Purification Process

hDectin1-Fc was purified using a GE Akta Purifier running a Unicorn 5 operating system. The culture media was filtered with 0.22 micron filter to remove particulates before being loaded at a rate of 5 ml/min into a column with TOSOH Protein A resin. The column was washed with 3 column volumes of pH 7 PBS buffer then 2 column volumes of 2M NaCl to remove unspecific binders followed by another 2 column volumes of pH7 PBS to wash out the salt. Elution was done at 5 ml/min of pH4.5 Acetic acid and collected in a mechanical fractionator at 1.5 ml per well. The wells containing hDectin1-Fc based on the chromatogram were pooled, neutralised with Tris (Sigma-Aldrich, St Louis, Mo. USA) and concentrated with Merck Amicon Ultra-15 Centrifugal Filter Unit 10 kDa or 50 kDa molecular weight cut off as per manufacturer's protocol. The concentrated sample was then loaded on the GE Akta Explorer superloop and injected into the GE Healthcare HiLoad 16/600 Superdex 200 pg size exclusion chromatography column. The column was ran in pH7 PBS buffer at 1 ml/min and the fractions collected at in 1.5 ml per well by a mechanical fractionator.

In Vitro Co-Culture CFU Assay

Macrophage assay: Human peripheral blood CD14+ monocytes were cultured at 1×106 cells/mL in 5 mL of ImmunoCult™-SF Macrophage Differentiation Medium with Human Recombinant M-CSF at 50 ng/ml in a T-25 flask at 37° C. in a 5% CO₂ incubator. On Day 4, 2.5 mL of fresh ImmunoCult™-SF Macrophage Differentiation Medium was added to the flask. On Day 6, M1 activation was initiated with the addition of 50 ng/mL IFN-γ. On Day 8, the macrophages were treated with ACCUTASE and scrapped from the flask. The cells were centrifuged and resuspended in RPMI medium with 10% FBS and seeded into Eppendorf 96 well plates at 10⁵ cells/well.

NK cell assay: Human Peripheral Blood CD56+NK Cells 1×10⁶ cells/mL in 5 mL of ImmunoCult™-XF T Cell Expansion Medium with Human Recombinant IL-2 at 500 IU/ml in a T-25 flask at 37° C. in a 5% CO₂ incubator for 7 days. Additional fresh media was added on day 4. The cells were centrifuged and resuspended in RPMI medium with 10% FBS and seeded into Eppendorf 96 well plates at 10⁵ cells/well.

Monocyte assay: Human peripheral blood CD14+ Monocytes were cultured at 1×10⁶ cells/mL in 5 mL of ImmunoCult™-SF Macrophage Differentiation Medium with Human Recombinant M-CSF at 10 ng/ml in a T-25 flask at 37° C. in a 5% CO₂ incubator for 4 days. The cells were centrifuged and resuspended in RPMI medium with 10% FBS and seeded into Eppendorf 96 well plates at 10⁵ cells/well.

Neutrophil assay: Human peripheral neutrophils were extracted from Human peripheral blood mononuclear cells using EasySep™ Direct Human Neutrophil Isolation Kit as per manufacturer's protocol. The isolated neutrophils were centrifuged and resuspended in RPMI medium with 10% FBS and G-CSF and seeded into Eppendorf 96 well plates at 10⁵ cells/well.

The co-culture CFU assay involves the addition of 10⁵ cells/well of SC5314 Candida albicans cells to wells containing 10⁵ cells/well of primary immune cells with final concentration of hDectin1-Fc at 0, 1, 10, 100 or 1000 g/ml in a 96 well plate. The assay was incubated for 1 hr at 37° C. in a 5% CO₂ atmosphere incubator. Samples were taken from each well at the end of the incubation and diluted accordingly before plating on YPD agar plates. The colonies were counted the following day. Each assay was done thrice and the average taken.

In Vitro Combination Therapy Assay

Human peripheral blood CD14+ monocytes were cultured at 1×10⁶ cells/mL in 5 mL of ImmunoCult™-SF Macrophage Differentiation Medium with Human Recombinant M-CSF at 50 ng/ml in a T-25 flask at 37° C. in a 5% CO₂ incubator. On Day 4, 2.5 mL of fresh ImmunoCult™-SF Macrophage Differentiation Medium was added to the flask. On Day 6, M1 activation was initiated with the addition of 50 ng/mL IFN-γ. On Day 8, the macrophages were treated with ACCUTASE and scrapped from the flask. The cells were centrifuged and resuspended in RPMI medium with 10% FBS and seeded into 96 well plates at 10⁵ cells/well. The co-culture assay involves the addition of 10⁵ cells/well of SC5314 Candida albicans cells to wells containing 10⁵ cells/well of human primary macrophages cells with final concentration of hDectin1-Fc at 0, 1, 10 or 100 g/ml and varying concentration of Amphotericin B in a checkerboard dilution assay format in a 96 well plate. The assay was incubated for 24 hr at 37° C. in a 5% CO₂ atmosphere incubator and the MIC and MFC determined visual using a Nikon Eclipse Ti-E inverted microscope.

In Vivo Pharmacokinetics Analysis

9 week old female Balb/c mice (InVivos, Singapore) were injected intraperitoneally with 0.5, 1, 2 or 4 mg hDectin1-Fc in groups of 3. Blood was sampled from the tail at time 0, 5, 10, 15, 30 mins and 1, 2, 4, 8 hours thereafter followed by daily sampling with Microvette® (SARSTEDT, Nümbrecht, Germany) for 20 days. The respective blood samples were allowed to clot and centrifuged at 2000 g and the blood serum collected. The blood serum was diluted accordingly and the concentration of hDectin1-Fc was tested with Human IgG ELISA Antibody Pair Kit and developed with pNPP ELISA Substrate (STEMCELL Technologies, Vancouver, Canada). The plates were read on PLATE reader and the data analysed for hDectin1-Fc pharmacokinetic parameters with open source Microsoft Excel add-in PKSolver (China Pharmaceutical University, Nanjing, China).

In Vivo Mouse Monotherapy Survival Model

9 week old female Balb/c mice (InVivos, Singapore) were injected intraperitoneally with 1, 2 or 4 mg hDectin1-Fc in groups of 8 and the drug was allowed to distribute within the mice till its peak concentration at 2 hours based on the pharmacokinetic data. The mice were then injected intravenously with 0.5, 0.25 or 0.1 milion SC5314 Candida albicans cells to achieve a haematogenously disseminated candidiasis model. Another set of mice were injected with 0.5, 1, 2 mg hDectin1-Fc followed by intravenous injection with SC5314 Candida albicans 2 hours later. Each Candida albicans inoculum experimental set had an untreated group of 8 mice that were only injected intravenously with Candida albicans. The mice were observed daily and moribund mice were euthanized and counted as dead the following day. Kaplan-Meier survival plots were made to track the survival overtime of the mice. Statistical analysis of the difference between mice treated with hDectin1-Fc or untreated was done with the Mantel-Cox log rank test.

In Vivo Mouse Combination Therapy

The combination therapy group with 9 week old female Balb/c mice (InVivos, Singapore) were injected intraperitoneally with a single dose of 1 mg hDectin1-Fc in groups of 8 and the drug was allowed to distribute within the mice till its peak concentration at 2 hours based on the pharmacokinetic data. They were then injected intravenously with 0.5 milion SC5314 Candida albicans cells to achieve a haematogenously disseminated candidiasis model followed by Amphotericin B deoxycholate (Merck Sigma-aldrich Darmstadt, Germany) at 0.05 mg/kg/day intraperitoneally for 7 days. A monotherapy group of eight 9 week old female Balb/c mice (InVivos, Singapore) were injected intravenously with 0.5 milion SC5314 Candida albicans cells to achieve a haematogenously disseminated candidiasis model followed by Amphotericin B deoxycholate at 0.05 mg/kg/day intraperitoneally for 7 days. A monotherapy group of eight 9 week old female Balb/c mice (InVivos, Singapore) were injected intraperitoneally with a single dose of 1 mg hDectin1-Fc and the drug was allowed to distribute within the mice till its peak concentration at 2 hours then injected intravenously with 0.5 milion SC5314 Candida albicans cells. An untreated group was injected intravenously with 0.5 milion SC5314 Candida albicans cells. The four groups of mice were observed daily and moribund mice were euthanized and counted as dead the following day. Kaplan-Meier survival plots were made to track the survival overtime of the mice. Statistical analysis of the difference between mice treated with hDectin1-Fc or untreated was done with the Mantel-Cox log rank test.

Materials & Methods: hDectin1-AmB

Recombinant Plasmid Cloning

The pUC57 plasmids containing genetic sequence of nHis-hDectin1(A), nHis-hDectin1(B), nHis-hDectin1(C) and cHis-hDectin1(A), cHis-hDectin1(B), cHis-hDectin1(C) were bought (Genscript, Nanjing, China). These plasmids were transformed respectively into One Shot™ TOP10 Chemically Competent E. coli (Invitrogen™, Waltham, Mass. USA) according to the manufacturer's protocol and propagated overnight at 37° C. The plasmids were extracted and purified the following day using NucleoBond® Xtra Midi kit (Macherey-Nagel, Duren, Germany). The gene of interest of each plasmid was cut from the respective pUC57 plasmid with restriction enzymes NheI and EcoRI (New England Biolabs, Ipswich, Mass. USA) and the digested mixture ran on a electrophoresis gel. The band containing the gene of interest was excised and purified using NucleoSpin® Gel and PCR Clean-up kit (Macherey-Nagel, Duren, Germany). The gene of interest was then ligated with T4 DNA Ligase (New England Biolabs, Ipswich, Mass. USA) with an in-house vector backbone containing the Zeocin resistance gene or DHFR enzyme selection marker gene. The newly ligated plasmids were then transformed into One Shot™ TOP10 Chemically Competent E. coli (Invitrogen™, Waltham, Mass. USA), propagated overnight and extracted similarly. The purified plasmids were then linearized with BstBI (New England Biolabs, Ipswich, Mass. USA) and ethanol precipitated in preparation for transfection into Chinese Hamster Ovary (CHO) K1 or DG44 cells. The plasmids containing nHis-hDectin1(A)-Fc and nHis-hDectin1(B)-Fc were cloned in the same process as mentioned above.

Vector Screening & Production Cell Vehicle Comparison

The zeocin resistant gene plasmids of nHis-hDectin1(A), nHis-hDectin1(B), nHis-hDectin1(C) and cHis-hDectin1(A), cHis-hDectin1(B), cHis-hDectin1(C) prepared previously were transfected into CHO K1 cells using the Amaxa SG Cell Line 4D-Nucleofector™ X kit with the 4D-Nucleofector™ System (Lonza, Basel, Switzerland) at 10⁷ cells/ml and 4 g of each plasmid respectively. Cells in HyClone PF CHO media (GE Healthcare, Chicago, Ill. USA) were placed in static culture in a 37° C. incubator with a 5% CO₂ atmosphere for 48 hours before being transferred to a 96 well plate at 10⁴ cells/well in HyClone PF CHO media with 600 g/ml Zeocin (Gibco, Carlsbad, Calif. USA). The cells were regularly observed under a Nikon Eclipse Ti-E inverted microscope to identify surviving cell pools and to select confluent wells. Media from confluent wells were collected and used for subsequent Western blot analysis to determine the best expressed hDectin1 fragment. DHFR gene plasmid of nHis-hDectin1(A) was similarly transfected into CHO DG44 cells in the process mention above. Cells after transfection were transferred to a 96 well plate at 10⁴ cells/well in HyClone PF CHO media without Hypoxantine, Thymidine and Glycine (−)HT. The transfected CHO DG44 cells that survive the (−)HT were transferred to shake flask culture and subjected at stepwise increasing concentrations of Methotrexate (Merck Sigma Aldrich, Darmstadt, Germany) of 50, 150 and 250 nM. At each concentration of methotrexate, the cells were cultured till their viability improves back to 95%.

Synthesis of N-linked & C-linked methyl terminal PEG Amphotericin B (AmB)

To a solution 0.5 ml DMF (Merck, Darmstadt, Germany) at r.t.p, 0.005411 mmol Amphotericin B (Sigma-Aldrich, St. Louis, Mo. USA) and 0.00595 mmol MS(PEG)₁₂ (Thermo Scientific, Rockford, Ill. USA) were added in a 1:1.2 mole ratio and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (4CHCl₃: 1MeOH: 0.1H₂O) to give N-AmBPEG₁₂CH₃·N-AmBPEG₄CH₃ and N-AmBPEG₂₄CH₃ was synthesized with the same reagent mole ratio and purified by flash chromatography (4CHCl₃: 1MeOH: 0.1H₂O).

Synthesis of C-linked methyl terminal PEG Amphotericin B (AmB)

To a solution 1.5 ml DMF (Merck, Darmstadt, Germany) at r.t.p, 0.029 mmol Amphotericin B (Sigma-Aldrich, St. Louis, Mo. USA) and 0.044 mmol Fmoc-Cl (Sigma-Aldrich, St. Louis, Mo. USA) were added and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (10CHCl₃: 4MeOH: 0.3H₂O) to give FmocAmB.

To a solution 0.5 ml DMF (Merck, Darmstadt, Germany) at r.t.p, 0.00436 mmol FmocAmB, 0.0131 mmol EDC.HCl (Thermo Scientific, Rockford, Ill. USA) and 0.0131 mmol NHS (Thermo Scientific, Rockford, Ill. USA) were added and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (6CHCl₃: 1MeOH: 0.1H2O) to give FmocAmBNHS.

To a solution 0.5 ml DMF (Merck, Darmstadt, Germany) at r.t.p, 0.008847 mmol FmocAmBNHS and 0.0177 mmol MA(PEG)₁₂ (Thermo Scientific, Rockford, Ill. USA) were added in a 1:2 mole ratio and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (4CHCl₃: 1MeOH: 0.1H₂O) to give FmocAmBPEG₁₂CH₃. FmocAmBPEG₄CH₃ was synthesized with the same reagent mole ratio and purified by flash chromatography (3CHCl₃: 1MeOH: 0.15H₂O). FmocAmBPEG₂₄CH₃ was synthesized with the same reagent mole ratio and purified by flash chromatography (6CHCl₃: 1MeOH: 0.1H₂O).

Synthesis of C-Linked Maleimide Terminal PEG Amphotericin B (AmB)

To a solution 1.5 ml DMF (Merck, Darmstadt, Germany) at r.t.p, 0.029 mmol Amphotericin B (Sigma-Aldrich, St. Louis, Mo. USA) and 0.044 mmol Fmoc-Cl (Sigma-Aldrich, St. Louis, Mo. USA) were added and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (10 CHCl₃: 4MeOH: 0.3H₂O) to give FmocAmB.

To a solution 0.5 ml DMF (Merck, Darmstadt, Germany) at r.t.p, 0.00436 mmol FmocAmB, 0.0131 mmol EDC.HCl (Thermo Scientific, Rockford, Ill. USA) and 0.0131 mmol NHS (Thermo Scientific, Rockford, Ill. USA) were added and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (6CHCl₃: 1MeOH: 0.1H₂O) to give FmocAmBNHS.

To a solution 0.3 ml DMF (Merck, Darmstadt, Germany) at r.t.p, 0.00161 mmol FmocAmBNHS and 0.0199 mmol CA(PEG)₁₂ (Thermo Scientific, Rockford, Ill. USA) were added and the reaction stirred for 48 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography by step elution to remove impurities first (10 CHCl₃: 4MeOH: 0.3H₂O) then the product eluted (10 CHCl₃: 5MeOH 0.5H₂O) with tailing to give FmocAmBPEG₁₂COOH.

To a solution 0.5 ml DMF (Merck, Darmstadt, Germany) at r.t.p, 0.01088 mmol FmocAmBPEG₁₂COOH, 0.03265 mmol EDC.HCl (Thermo Scientific, Rockford, Ill. USA), 0.03265 mmol HOBt (Sigma-Aldrich, St. Louis, Mo. USA), 0.03265 mmol N-(2-aminoethyl)maleimide HCl (Sigma-Aldrich, St. Louis, Mo. USA) and 0.03265 mmol (Sigma-Aldrich, St. Louis, Mo. USA) trimethylamine were added and the reaction stirred for 24 hrs. The reaction mixture was then precipitated in 15 ml diethyl ether (Merck, Darmstadt, Germany) and the solvent decanted after centrifugation at 3000 rpm for 5 minutes. The crude mixture was purified by flash chromatography (4CHCl₃: 1MeOH: 0.1H₂O) to give FmocAmBPEG₁₂Mal.

### Example 1

Screening of hDectin1 Fragments

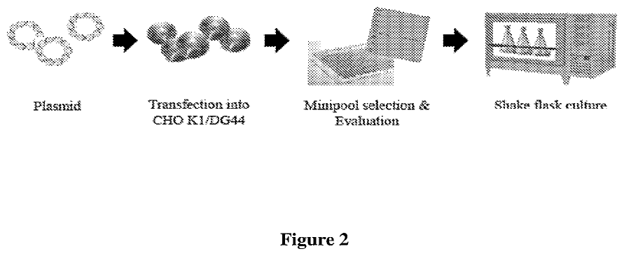

Three random truncations of hDectin1 fragments from the extracellular domain to just before the transmembrane domain of were designed: hDectin1(A), hDectin1(B), hDectin1(C). In addition, each of these was made to have an N-terminus or C terminus Histag. This gave a total array of 6 different variants to screen: nHis-hDectin1(A), nHis-hDectin1(B), nHis-hDectin1(C) and cHis-hDectin1(A), cHis-hDectin1(B), cHis-hDectin1(C). Each of these variants was developed according to the process flow shown in FIG. 2. The plasmids for nHis-hDectin1(A), nHis-hDectin1(B), nHis-hDectin1(C) and cHis-hDectin1(A), cHis-hDectin1(B), cHis-hDectin1(C) plasmid vectors were transfected into CHO K1 cells respectively and put through zeocin selection. The surviving polyclonal minipools were then evaluated by western blot.

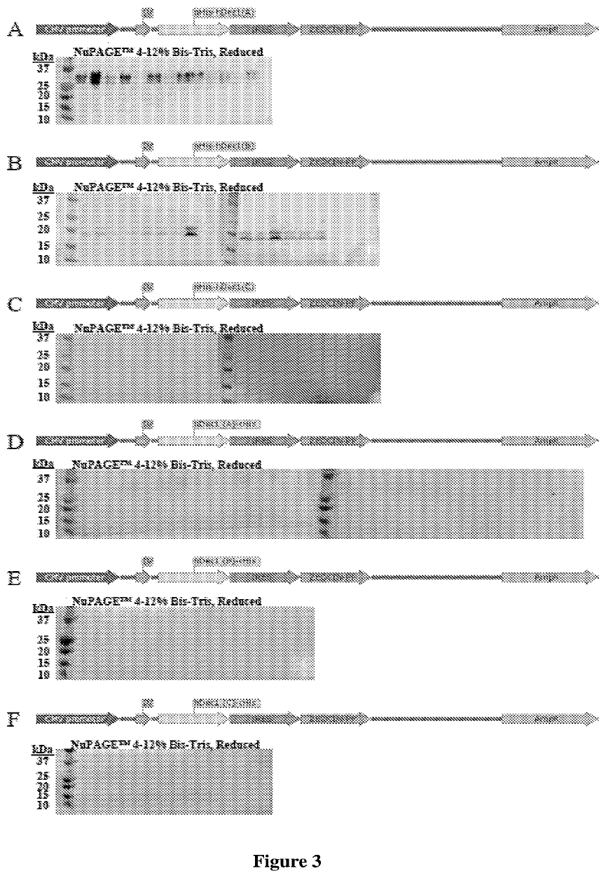

The general results of the Western Blot screening (FIG. 3) suggest that expression for the hDectin1-cHis variants were visually absent and deemed to be non-existent or appreciably low. The Western Blots for the nHis-hDectin1(A) variants showed nHis-hDectin1(A) and nHis-hDectin1(B) to be able to express hDectin1. nHis-hDectin1(A) and nHis-hDectin1(B) were then used as the fragment for further construction into hDectin1-Fc.

Screening of hDectin1-Fc Constructs

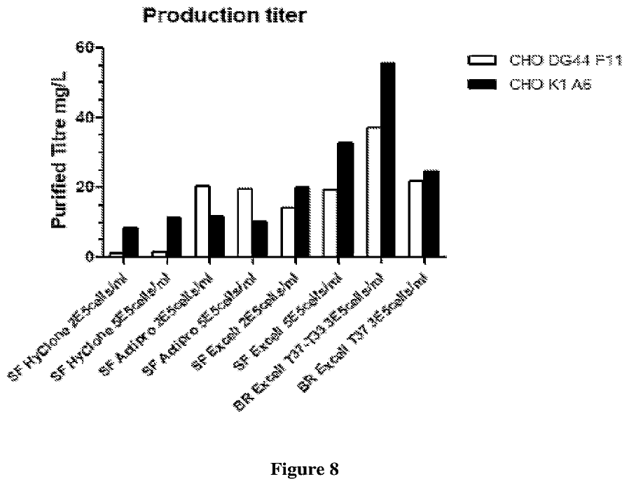

The genetic sequence for nHis-hDectin1(A) and nHis-hDectin1(B) fragment variants were combined respectively with the genetic sequence of the Fragment Constant (Fc) from a Human Immunoglobulin G1 to express them as the Fc-fusion proteins nHis-hDectin1(A)-Fc and nHis-hDectin1(B)-Fc. Each of these variants were developed according to the process flow shown in FIG. 8. nHis-hDectin1(A)-Fc and nHis-hDectin1(B)-Fc plasmid vectors were transfected into CHO K1 cells respectively and put through zeocin selection. The surviving polyclonal minipools were then evaluated by western blot.

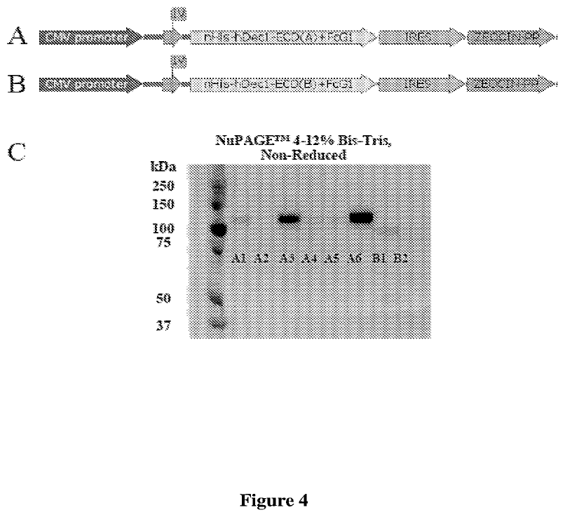

The Western Blot screen of nHis-hDectin1(A)-Fc and nHis-hDectin1(B)-Fc yielded lesser surviving cell minipools but with nHis-hDectin1(A)-Fc having two distinctly better cell pools (FIG. 4). nHis-hDectin1(A)-Fc was therefore determined to be the best candidate to use for further development.

Recombinant Production of hDectin1-Fc

Production Cell Vehicle Comparison

As mammalian expression systems continue their increasing trend of being favoured over non-mammalian ones, Chinese Hamster Ovary (CHO) cell-based systems maintain their dominance in the category of production of Fc containing biopharmaceuticals proteins such as monoclonal antibodies and Fc-fusion proteins. The popularity of CHO cell expression systems lie in their ability to express the target protein by gene amplification, to fold expressed proteins correctly and to add human compatible mammalian post translational glycoforms. The two common mammalian Chinese Hamster Ovary Cell (CHO) types used for recombinant protein production are CHO K1 and CHO DG44. Both CHO cell types can be cultured in adherent or suspension mode though suspension culture is favoured for scaling purposes. CHO K1 is a genetically intact cell line while CHO DG44 is deficient in the DHFR enzyme. This translates to positively transfected CHO K1 cells being selected by survival in Zeocin antibiotic medium and positively transfected CHO DG44 cells by survival in Hypoxanthine, Thymidine and Glycine deficient medium in the presence of Methotrexate.

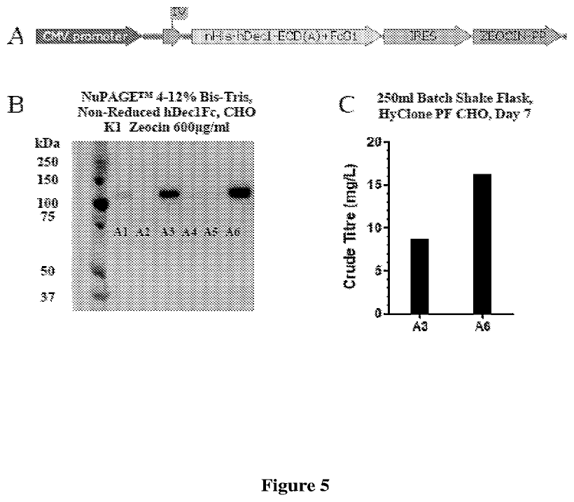

Selection of positively transfected CHO K1 cells occurs in a single step by culturing in media at a concentration of 600 μg/ml of Zeocin. The “kill-concentration” of 600 μg/ml was determined in another experiment where untransfected CHO K1 cells were subjected to different Zeocin concentrations. For CHO K1, minipools A3 and A6 were determined to be able to express nHis-hDectin1(A)-Fc from the Western Blot with A6 being the best producer from the preliminary 7 day culture run. The titers of the producing minipools were evaluated by ELISA after a 7 day culture and minipool A6 was identified as the higher producer (FIG. 5).

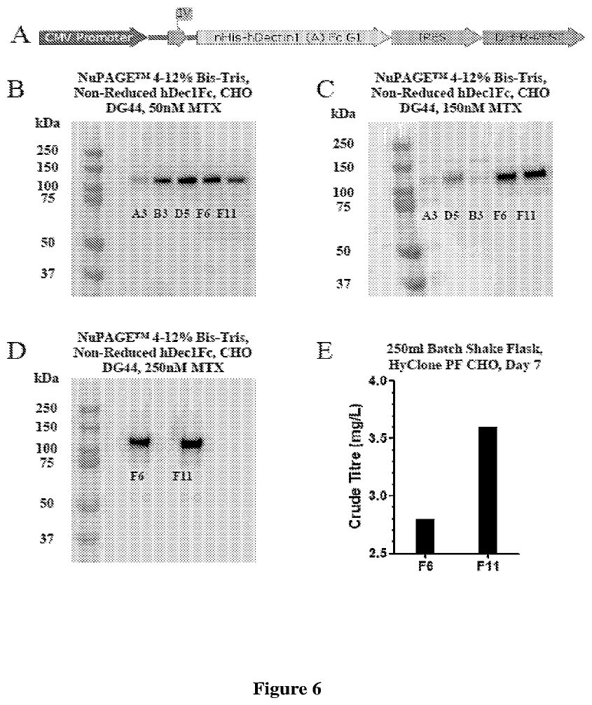

Positively transfected CHO DG44 cells were selected through a stepwise amplification process in media deficient in Hypoxanthine, Thymidine and Glycine and increasing concentrations of Methotrexate. Western blot was used to monitor the expression of nHis-hDectin1(A)-Fc at each concentration of Methotrexate. The final two minipools that survive the selection process are F6 and F11. The 7 day preliminary culture runs evaluated by ELISA showed F11 to be the better candidate for CHO DG44 (FIG. 6). Comparison of the 7 day culture best expressing candidates for both positively transfected CHO K1 and CHO DG44, it is apparent that the CHO K1 cell line is able to produce nHis-hDectin1(A)-Fc better than CHO DG44.

Optimization of Culture Conditions

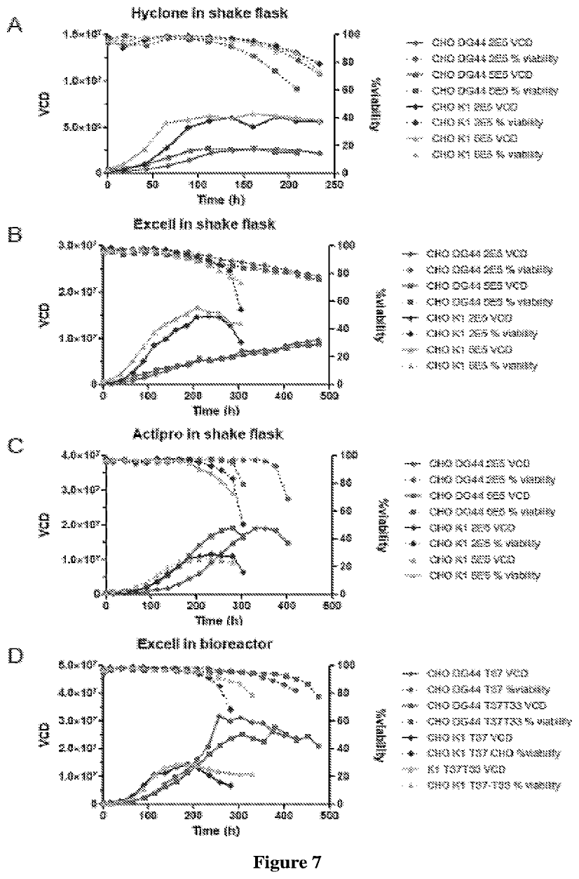

Optimal production of secreted recombinant hDectin1-Fc in Chinese Hamster Ovary Cells (CHO) is a balance between production cell vehicle and culture conditions like media, seeding density and temperature. In the previous section comparing production cell vehicles, the cell pool F11 made from CHO DG44 and A6 made from CHO K1 were determined to be the top producers of hDectin1-Fc for their respective cell lines. CHO K1-A6 showed a higher titer than CHO DG44-F11. To further study the influence of cell culture media on the growth profile and protein titer of CHO DG44-F11 and CHO K1-A6, three commercially available mammalian cell culture media Hyclone, Excell and Actipro were selected and the respective cell pools seeded at 2×10⁵ cells/ml or 5×10⁵ cells/ml in 2 L shake flasks and cultured at 37° C. (FIG. 7A-C). The cell density and viability were monitored daily and the cultures terminated when % viability dropped pass 70%.

The results of the study revealed Excell and Actipro to be able to facilitate high peak density cell growth between 1-2×10⁷ cells/ml with Excell favouring CHO K1 growth while Actipro favoured CHO DG44 growth (FIG. 7B, 7C). Both Excell and Actipro outperform Hyclone in terms of peak cell density and culture duration (FIG. 7A-C). In Excell media, CHO K1 grow at a faster rate than CHO DG44 with a higher seeding density generally giving marginally higher cell density across the culture duration (FIG. 7B). A higher seeding density also translated into higher titer for Excell media cultures though CHO K1 A6 produced more hDectin1-Fc (FIG. 8). Actipro media favoured CHO DG 44 growth but on the contrary, a lower seeding density results in delayed time to peak cell density and longer culture duration thought the peak cell density remains unaffected (FIG. 7C). CHO K1 A6 growing in Excell media and seeded at 5×105 cells/ml however produced a much higher titer amongst the shake flask cultures. Taking into account rate of cell growth, peak cell density, viable culture duration and titer, Excell media was chosen to be the optimal medium for producing hDectin1-Fc.

To further evaluate production of hDectin1-Fc at a scale appropriate for sustaining subsequent in vitro and in vivo studies, CHO DG44-F11 and CHO K1-A6 were cultured in 5 L bioreactors in Excell media and seeded at a cell density of 3×10⁵ cells/ml in fed-batch mode. The pH was maintained at 7 and the temperature at 37° C. in a constant temperature run or lowered from 37° C. to 33° C. at peak cell density in a temperature shift run. The controlled temperature and pH environment of the bioreactor permitted better cell growth with longer sustained % viability for both CHO DG44-F11 and CHO K1-A6. The peak cell density of CHO DG44-F11 was twice that of CHO K1-A6 (FIG. 7D). However the titer of both CHO DG44-F11 and CHO K1-A6 grown at 37° C. constant temperature in the bioreactor did not outperform the shake flask culture. The bioreactor culture grown at 37° C. and temperature shifted to 33° C. when peak cell density was achieved had prolonged high % viability and significantly higher titer for CHO K1-A6 (FIG. 8). It is worth noting that the peak cell density of CHO K1-A6 in shake flask or bioreactor culture is similar but the controlled environment of a temperature shifted bioreactor promoted higher production of hDectin1-Fc. The overall results led to the conclusion that CHO K1-A6 grown in Excell media in a fed-batch bioreactor with temperature shift at peak cell density is the most optimal for producing hDectin1-Fc.

Evaluation of hDectin1-Fc Structure & Functionality

Western Blot Analysis of hDectin1-Fe

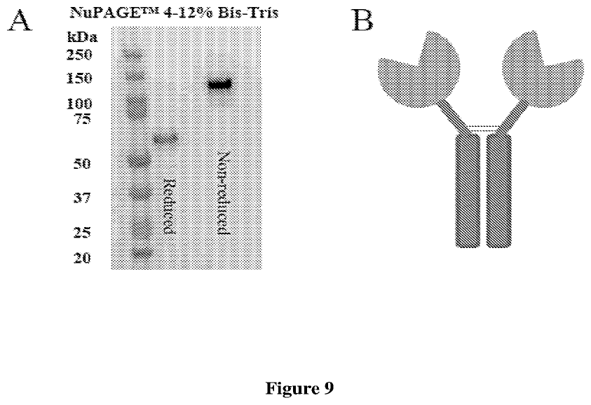

To evaluate whether the designed hDectin1-Fc fusion protein was stably produced and secreted by Chinese Hamster Ovarian (CHO) cells, a denaturing Western Blot was ran both under reducing conditions and non-reducing conditions. The band observed on the Western Blot (FIG. 9A) under reducing conditions was detected to be between the 50 kDa and 75 kDa bands on the protein ladder with the expected mass to be 49.5 kDa. The difference is likely due to the presence of glycan chains on the protein. The non-reduced band is between 100 kDa and 150 kDa, suggesting that the protein is a dimer; in line with expectations based on the presence of disulphide linkages on the Fc portion. hDectin1-Fc is suggested to have a structure (FIG. 9B) similar to an antibody but with the top half made of hDectin1 instead.

Immunofluorescence Assay: hDectin1-Fc Binding to Candida albicans

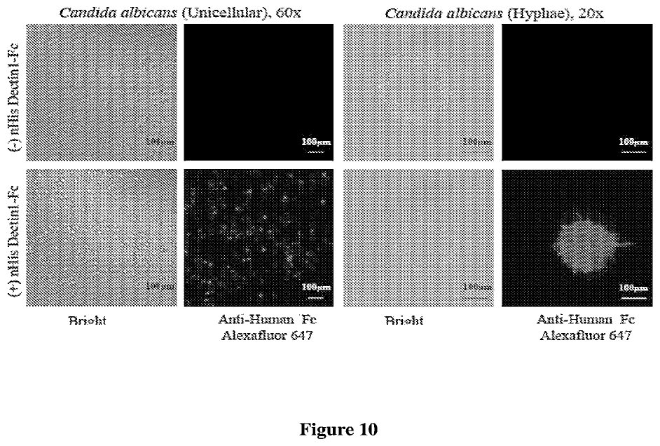

The hDectin1-Fc protein was assayed via immunofluorescence to verify if the Dectin1 domain maintains it is ability to bind to β-glucans on yeast after fusion with human Immunoglobulin G's Fc (FIG. 10). The assay was ran on both unicellular yeast and hyphae morphologies of Candida albicans. The results show that red fluorescence from anti-human Fc conjugated Alexafluor 647 is observed in the presence of hDectin1-Fc for both unicellular and hyphae forms. It was also observed that only particular areas corresponding to exposed β-glucans on Candida albicans were fluorescing. The results of this assay demonstrated the ability of hDectin1-Fc to bind Candida albicans.

Surface Plasmon Resonance Assay: hDectin1-Fc Binding to Fc-Receptors

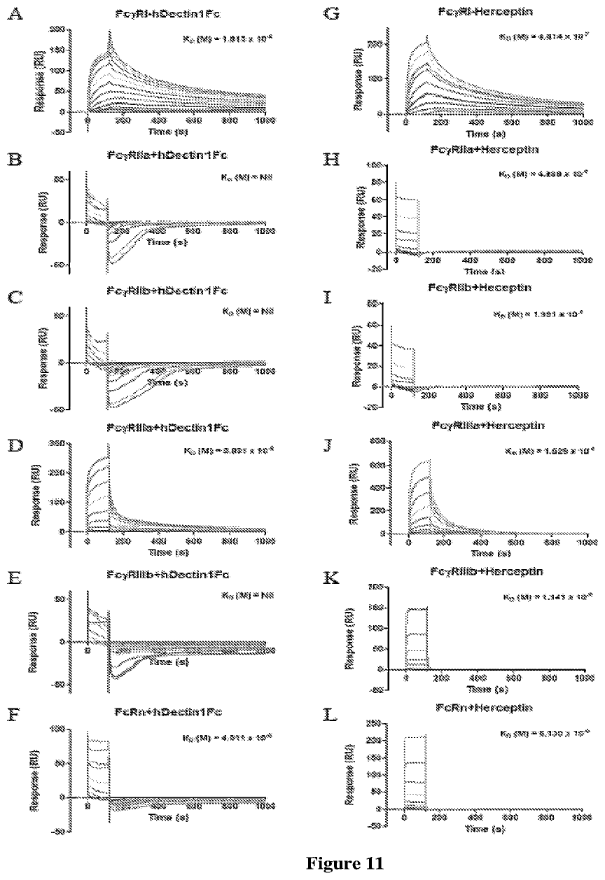

hDectin1-Fc was designed to mimic the functions of an Immunoglobulin G1(IgG1) antibody with its Dectin1 domain representing the antigen binding domain of an antibody and its Fc domain similar to that of an IgG1. Antibody mediated immune activation depends on the strength of binding and affinity of the Fc to the various Fcγ receptors. Binding to the neonatal receptor FcRn is also important for the half-life in serum and the transcytosis across endothelial cells into various tissues. Surface plasmon resonance was used to evaluate the binding of the Immunoglobulin G's Fc of hDectin1-Fc to the various Fc receptors (FcγRI, FcγRIIa, FcγRIIb, FcγRIIIa, FcγRIIIb and FcRn) present on immune cells by flowing free hDectin1-Fc of various concentrations over immobilised Fc receptors (FIG. 11A-F). Herceptin (Trastuzumab) an IgG1 antibody used against Her2 positive breast cancer was used as a comparision to benchmark the interaction of hDectin1-Fc as a Fc-fusion with Fc receptors compared to a full structure IgG1(FIG. 11G-L). The overall Binding Constant KD results of the screen revealed hDectin1-Fc to be able to interact with and bind to FcγRI, FcγRIIIa and FcRn only while Herceptin was able to bind to all the Fc receptors. High affinity FcγRI(CD64) is expressed on macrophages, monocytes and neutrophils and low affinity FcγRIIIa (CD16a) is expressed on natural killer cells, monocytes, macrophages and neutrophils. Both CD64 and CD16a activating receptors have Immunoreceptor Tyrosine based Activation Motif (ITAM) signalling domain. FcγRI strongly binds IgG opsonized targets to trigger phagocytosis and a proinflammatory response of TNFα, IFNγ and production of oxidative species. FcγRIIIa likewise activate degranulation, phagocytosis, and oxidative burst. The KD values of hDectin1-Fc and Herceptin binding to FcγRI, FcγRIIIa and FcRn generally show that the fusion protein has a moderately weaker binding.

In Vitro CFU Assay: Innate Immune Cells Against Candida albicans

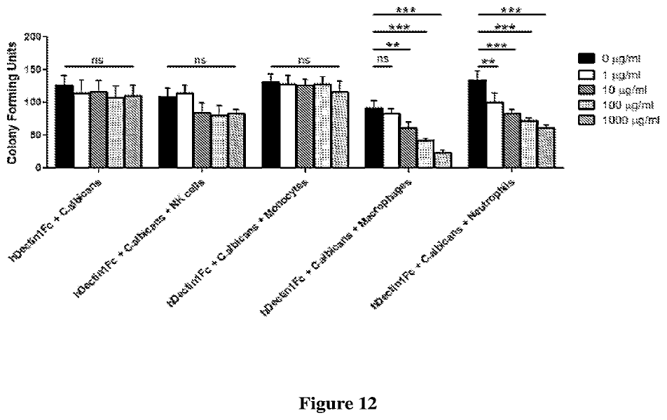

To evaluate the capability of the hDectin1-Fc fusion protein in enhancing immune cell anti-candida function, 10⁵ human primary immune cells were co-cultured with 10⁵ Candida albicans in a 1 hr long assay incubated at 37° C. with varying concentrations of hDectin1-Fc from 0-1000 g/ml. The surviving Candida cells were diluted and plated and the colony forming units (CFU) used as a measure of fungicidal activity (FIG. 12). The assay was not continued beyond 1 hour because of morphology change in the Candida albicans from unicellular to hyphae. NK cells, monocytes, macrophages and neutrophils are the immune cells implicated in anti-candida defence. In this assay short 1 hour assay, NK cells and monocytes did not demonstrate any appreciable fungicidal activity in the absence or presence of hDectin1-Fc. Macrophages and neutrophils however demonstrated a dose response with an inverse correlation between hDectin1-Fc dose and CFU. Between the hDectin1-Fc concentrations 0 μg/ml and 1000 g/ml is an approximate 50% reduction in CFU for both macrophages and neutrophils. Macrophages and neutrophils express on their cell surface the activating receptors FcγRI and FcγRIIIa which are responsible for activating phagocytic and fungicidal mechanisms. The previous surface plasmon resonance study showed the ability of hDectin1-Fc to bind to these two receptors, thereby explaining the observed outcome in macrophages and neutrophils. The low affinity FcγRIIIa receptor is also expressed on NK cells but while some degree of response was observed at the 10 g/ml and above doses, the overall result was not statistically significant. This could suggest that hDectin1-Fc's interaction with FcγRIIIa might not play an effective role in the duration of the assay or the fungicidal mechanism triggered may not be sufficiently potent.

In Vitro Combination Therapy Assay: Amphotericin B & hDectin1-Fc

Amphotericin B is a macrocyclic polyene antifungal drug made naturally by the bacterium Streptomyces nodosus. It binds preferentially to ergosterol in the fungus cell plasma membrane and extracts it; destabilising the membrane in the process and eventually leading to cell lysis.

The in vitro CFU assays with hDectin1-Fc, it was observed that the protein drug enhances phagocytosis but does not assist in total elimination of Candida albicans. To take advantage of hDectin1-Fc's ability to enhance phagocytosis, Amphotericin B and it were explored as a combination therapy to enhance total fungicidal activity against Candida albicans. 10′ Candida albicans cells and 10′ human primary macropahges were seeded per well in a checkerboard dilution assay of Amphotericin B with the respective concentration of hDectin1-Fc (0, 10, 100 μg/ml) and incubated for 24 hours at 37° C. The results of the assay (FIG. 13) show that between 0 μg/ml and 100 g/ml hDectin1-Fc is an approximate 45% reduction in MFC of Amphotericin B. At the hDectin1-Fc concentration of 100 g/ml, the MFC of Amphotericin B is 60 nM, which is below the basal MFC of 110 nM required to kill all the Candida albicans cells in the absence of hDectin1-Fc. This drop in the MFC is hypothesized to work by Amphotericin B weakening the fungus cell membrane and together with the enhanced phagocytosis facilitated by hDectin1-Fc, makes the fungus cell susceptible to the macrophage's fungicidal mechanisms upon phagocytosis. The Amphotericin B & hDectin1-Fc combination therapy demonstrates the possibility of marrying two different antifungal mechanisms to eliminate Candida albicans. While the reduction in MFC is not drastic and probably limited to the rate at which the macrophages can clear the fungus cells, it certainly does widen the therapeutic window in which Amphotericin B can be used in an in vivo setting.

In Vivo Pharmacokinetic Study of hDectin1-Fc

TABLE 2
Pharmacokinetic parameters for hDectin-Fc in mice (n = 3) at respective
single bolus doses and tracked over 20 days via blood serum sampling.

| Dose | [symbol] | t½ | Tₘₐₓ | Cₘₐₓ | AUC [symbol] | MRT₀₋ | Vz/F | Cl/F |
|---|---|---|---|---|---|---|---|---|
| (mg) | (1/h) | (h) | (h) | (μg/ml) | (μg/ml [symbol] h) | [symbol] (h) | (μg)/(μg/ml) | (μg)/(μg/ml)/h |
| 4 | 0.00898 | 77 | 2 | 1603 | 127191 | 120 | 3.5008 | 0.0314 |
| 2 | 0.00443 | 156 | 2 | 1039 | 210068 | 204 | 2.1489 | 0.0095 |
| 1 | 0.00474 | 146 | 2 | 294 | 61474 | 246 | 3.4254 | 0.0163 |
| 0.5 | 0.00692 | 100 | 10 | 330 | 76043 | 173 | 0.9495 | 0.0066 |

[symbol]

indicates data missing or illegible when filed

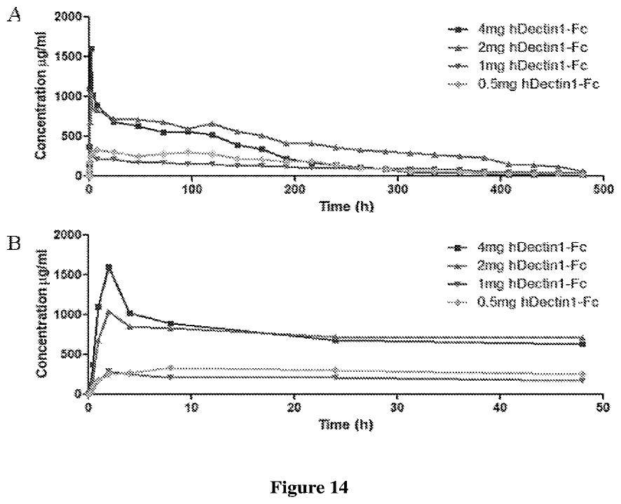

The pharmacokinetic parameters of hDectin1-Fc at different doses (4 mg, 2 mg, 1 mg and 0.5 mg) were determined by administering a single bolus intraperitoneal injection and sampling blood serum daily (FIG. 14). The daily concentration over 20 days was measured by ELISA and the results analysed with PKSolver to determine the pharmacokinetic parameters (Table 2). At a high dose of 4 mg, the drug hit a peak serum concentration of around 1600 μg/ml in two hours but was rapidly excreted given its lowest half-life and mean residence time (MRT) relative to the other doses. The 2 mg and 1 mg doses have a half-life about twice that of the 4 mg dose but the MRT of the 1 mg dose is the longest at 246 hours. The lowest dose of 0.5 mg has a half-life and MRT lower than the 2 mg and 1 mg dose and its peak concentration (Cmax) is at 10 hours instead of 2 hours like the other doses. This observed outcome for the 0.5 mg dose is likely to arise from the slow transfer of hDectin1-Fc from the intraperitoneal space of the mouse into its bloodstream due to the lower concentration gradient and stronger role of excretion over distribution. The duration of the MRT and the half-life is a reflection of the presence of the protective effects of hDectin1-Fc in the body of the mice. The optimal dose with the longest MRT and half-life is approximately 1 mg. The general take away from the pharmacokinetic study are: (i) high doses result in rapid excretion of the drug, (ii) peak concentration occurs at 2 hours (iii) lower doses have a longer half-life and mean residence time appropriate for passive immunization.

In Vivo Mouse Survival Assays: hDectin1-Fc Therapeutic Effect in Acute to Chronic Candida albicans Infection

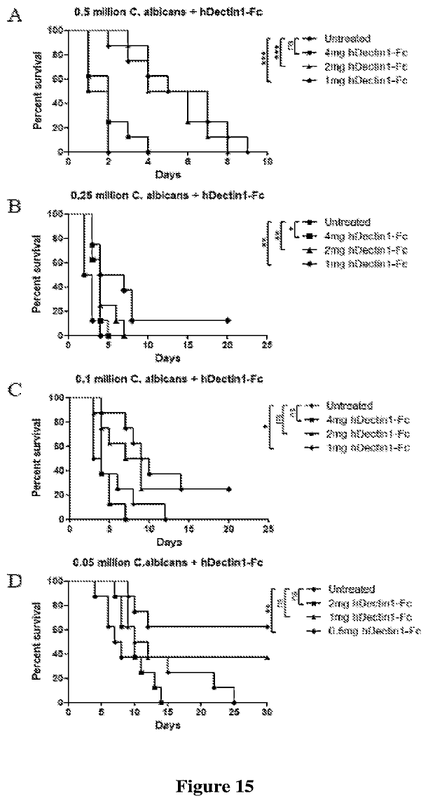

To examine the effect of hDectin1-Fc passive immunization against reducing the fatality of haematogenously disseminated candidiasis, a Balb/c murine model of hDectin1-Fc at various doses in relation to Candida albicans inoculum was used. Eight 9-week-old female Balb/c mice per treated group were passively immunized intraperitoneally with hDectin1-Fc at a specific dose (4 mg, 2 mg, 1 mg or 0.5 mg) and challenged with SC5314 Candida albicans (0.5 million, 0.25 million, 0.1 million and 0.05 million) intravenously after 2 hours. An untreated group without hDectin1-Fc but infected with the respective inoculum of Candida albicans was used as a control in each experimental set (FIG. 15). Survival in each group was monitored twice daily until all the mice had died or there was survival of 2 weeks from the last death. Moribund mice were euthanized and counted as part of the following day. The general trend amongst the experimental sets shows an inverse correlation between hDectin1-Fc dose and Candida albicans inoculum in relation to survival. The negative effect on survival of a high dose of hDectin1-Fc in relation to fungal inoculum is most apparent in the 4 mg hDectin1-Fc dose with 0.1 million Candida albicans (FIG. 15C) and 2 mg hDectin1-Fc with 0.05 million Candida albicans inoculum (FIG. 15D). In both groups, the rate of death was greater than the corresponding untreated group. In the lethal infection model at 0.5 million Candida albicans cells, the marginal optimal therapeutic effect of the 1 mg dose over the 2 mg dose can be observed by the closeness of the survival curves (FIG. 15A). The proximity between the 2 mg and 1 mg curves have a growing divergence as the fungal burden is reduced from 0.5 to 0.05 million (FIG. 15A-D). The most striking observation of optimal therapy is from the prominent shift in the survival curve of the group of mice treated with 0.05 mg hDectin1-Fc and infected at a subacute inoculum of 0.05 million Candida albicans cells. It can be concluded that hDectin1-Fc at the appropriate dose is able to improve survivability but endpoint survival improves as the Candida albicans inoculum decline from a lethal sepsis like infection to a slower sub-acute infection that fits the passive immunization therapeutic objective of hDectin1-Fc.

In Vivo Mouse Survival Assays: Amphotericin B & hDectin1-Fc

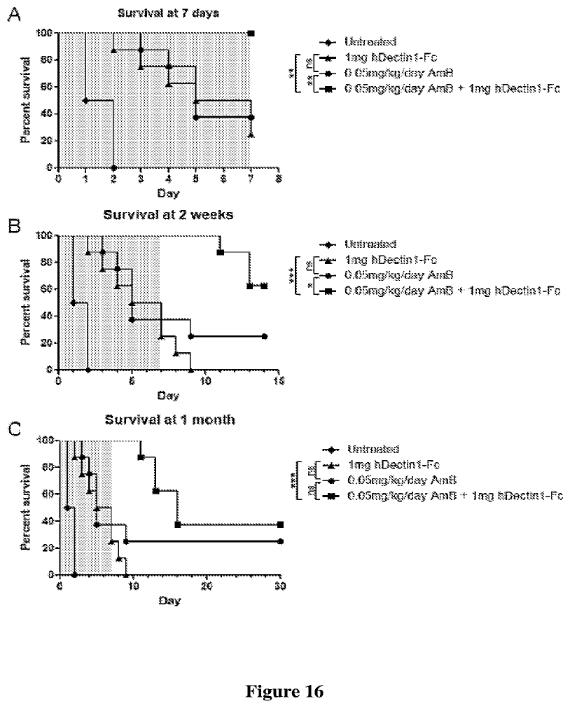

The previous 24 hour in vitro study of hDectin1-Fc and Amphotericin B with primary human macrophages against Candida albicans demonstrated a 45% reduction in the MFC level required to eliminate the fungus (FIG. 13). This suggested that combination therapy may have potential practical therapeutic use. To further develop upon the outcome of the in vitro combination therapy and to address the limitation of hDectin1-Fc monotherapy in treating an acute model of haematogenously disseminated candidiasis, an acute infection murine model using hDectin1-Fc passive immunization with Amphotericin B at sub-optimal dosing was used to study the therapeutic efficacy of an antibody-small molecule combination. Eight 9-week-old Balb/c mice were passively immunized using 1 mg hDectin1-Fc intraperitoneal single bolus dose and infected with 0.5 million Candida albicans SC5314. Amphotericin B was administered intraperitoneally post candida infection at 0.05 mg/kg/day for 7 days. The combination therapy groups were compared against monotherapy groups of 1 mg hDectin1-Fc single bolus dose and 0.05 mg/kg/day Amphotericin B for 7 days in eight 9-week-old mice per group infected with 0.5 million Candida albicans SC5314. An untreated group of eight 9-week-old mice infected with 0.5 million Candida albicans SC5314 was also included for reference. The outcome of the murine in vivo survival model for combination therapy in an acute infection demonstrated the superiority of the combination therapy over the individual monotherapy (FIG. 16). Mice in the combination therapy group were all healthy while mice in the untreated group were all dead by the second day while those in the monotherapy groups had fewer than 40% survival (FIG. 16A). After the cessation of treatment, it was observed that mice in the monotherapy groups continue to decline in their percentage survival while mice in the combination therapy group experienced a much delayed decline beginning only day 12 (FIG. 16B). The endpoint survival at 1 month post infection shows the difference between combination therapy and monotherapy declining to a non-significant difference.

### Example 2

Construction of hDectin1-AmB

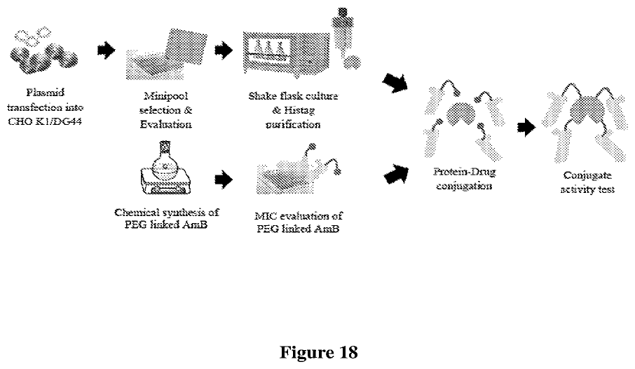

The developmental process of hDectin1-AmB (FIG. 18) is a convergent synthetic process involving the recombinant production of hDectin1 in mammalian CHO cells and the chemical synthesis of Amphotericin B with polyethylene glycol (PEG) linkers and subsequent conjugation of both entities.

Using protein databases such as Uniprot and RCSB Protein Data Bank, structural information for hDectin1 can be obtained and used as a reference as to where truncations can be made between the ectodomain and transmembrane domain of hDectin1. In general, the candidates of hDectin1 will consist of various truncations ideally at the portion of the protein that is a bend or fold and not regions with secondary structures like α-helixes or β-pleated sheets. The recombinant hDectin1 has to be stable in solution and able to bind to fungal β-glucans.

The conjugation of PEG as a linker to Amphotericin B and hDectin1 is a more complex process several interrelated factors such as synthetic feasibility, steric effect on activity and site of conjugation to consider. Amphotericin B has a variety of reactive organic functional groups and the synthesis route to connect the PEG linker to particular sites have to be synthetic feasible in terms of selectivity, yield and by-products. By conjugating a PEG linker to Amphotericin B, the original pharmacological activity of the drug could be altered as a result of new preferred conformations which may affect the interaction between Amphotericin B and its target fungal ergosterol. Lastly, conjugation of the Amphotericin B via PEG to hDectin1 has to consider the sites of conjugation on the protein and the drug to protein ratio such that the binding of hDectin1 to fungal β-glucans is not hindered.

Recombinant Production of hDectin1

Mammalian expression systems are favoured over non-mammalian for the development of human use biopharmaceuticals because of their ability to express the target protein by gene amplification, fold expressed proteins correctly and to add human compatible mammalian post translational glycoforms. Chinese Hamster Ovary (CHO) cell-based systems maintain their dominance in the category of production of Fc containing biopharmaceuticals proteins such as recombinant clotting factors, monoclonal antibodies and Fc-fusion proteins. The two common mammalian Chinese Hamster Ovary Cell (CHO) types used for recombinant protein production are CHO K1 and CHO DG44. Both CHO cell types can be cultured in adherent or suspension mode though suspension culture is favoured for scaling purposes. CHO K1 is a genetically intact cell line while CHO DG44 is deficient in the DHFR enzyme. This translates to positively transfected CHO K1 cells being selected by survival in Zeocin antibiotic medium and positively transfected CHO DG44 cells by survival in Hypoxanthine, Thymidine and Glycine deficient medium in the presence of Methotrexate.

For the initial screen, three random truncations of hDectin1 fragments from the extracellular domain to just before the transmembrane domain of were designed: hDectin1(A), hDectin1(B), hDectin1(C). In addition, each of these was made to have an N-terminus or C terminus Histag. This gave a total array of 6 different variants to screen: nHis-hDectin1(A), nHis-hDectin1(B), nHis-hDectin1(C) and cHis-hDectin1(A), cHis-hDectin1(B), cHis-hDectin1(C). Each of these variants was developed according to the process flow shown in FIG. 18. The plasmids for nHis-hDectin1(A), nHis-hDectin1(B), nHis-hDectin1(C) and cHis-hDectin1(A), cHis-hDectin1(B), cHis-hDectin1(C) plasmid vectors were transfected into CHO K1 cells respectively and put through zeocin selection. The surviving polyclonal minipools were then evaluated by western blot.

The general results of the Western Blot screening (FIG. 3) suggest that expression for the hDectin1-cHis variants were visually absent and deemed to be non-existent or appreciably low. The Western Blots for the nHis-hDectin variants showed nHis-hDectin1(A) and nHis-hDectin1(B) to be better expressed hDectin1. nHis-hDectin1(A) was chosen as the best candidate given the qualitatively higher frequency of expressing cell pools with darker bands on the Western Blot.

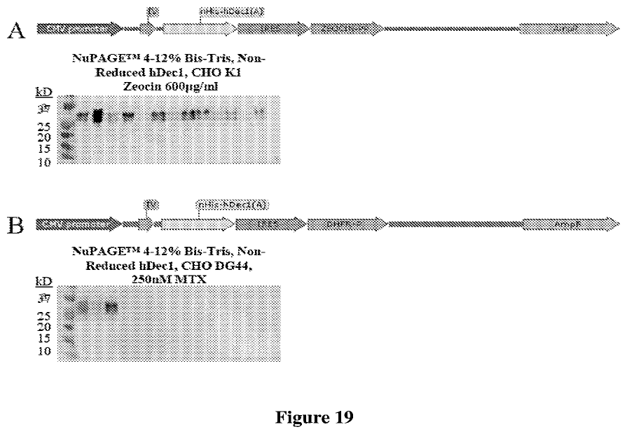

Selection of positively transfected CHO K1 cells occurs in a single step by culturing in media at a concentration of 600 g/ml of Zeocin. The “kill-concentration” of 600 μg/ml was determined in another experiment where untransfected CHO K1 cells were subjected to different Zeocin concentrations. Positively transfected CHO DG44 cells were selected through a stepwise amplification process in media deficient in Hypoxanthine, Thymidine and Glycine and increasing concentrations of Methotrexate. To evaluate whether the designed hDectin1 protein was stably produced and secreted by Chinese Hamster Ovarian (CHO) cells, a Western Blot was ran under reducing conditions for the cell pools of CHO K1 and CHO DG44 at the end of the selection process. The band observed on the Western Blot (FIG. 19) under reducing conditions was detected to be between the 25 kDa and 37 kDa on the protein ladder with the expected mass to be 28 kDa and the excess mass from glycans. It is apparent that the CHO K1 cell line is able to produce nHis-hDectin1(A) better than CHO DG44.

Site Linkage Comparison of Polyethylene Glycol (PEG) with Amphotericin B

Synthesis Process

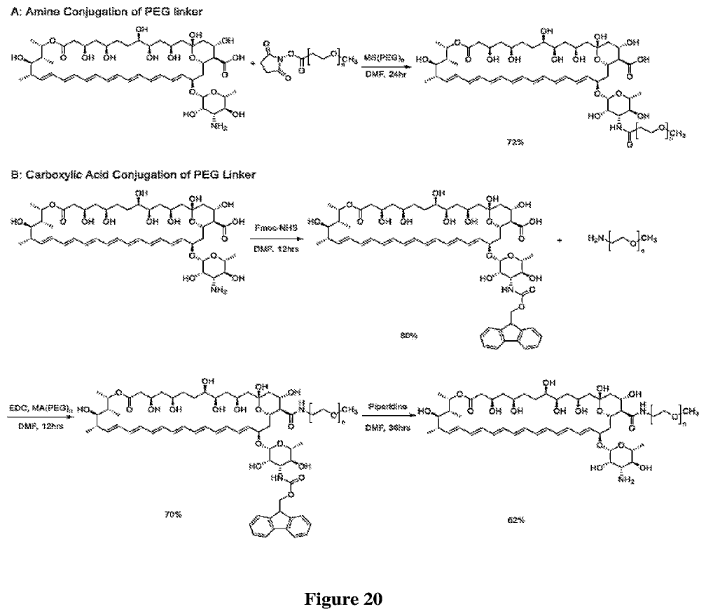

Linker conjugation site and its length can affect the activity of drug in the way it is able to interact with its target as a result of a change in preferred conformation. Amphotericin B has two possible sites of conjugation: the carboxylic acid on the macrocyclic ring (C-linked) and the amine on the sugar (N-linked). To investigate how conjugation to each of these sites affects the activity of Amphotericin B, polyethylene glycol (PEG) linkers of three different repeat units were attached to them (FIG. 20) and their MIC of Candida albicans evaluated against the unconjugated free Amphotericin B.

The conjugation of PEG linkers to the amine functional group of Amphotericin B is a one-step reaction that takes advantage of the higher reactivity of the amine with the NHS activated ester of the PEG linker compared to the much more abundant alcohol functional groups. Conjugating to the carboxylic acid of Amphotericin B requires the blocking of the amine with Fmoc followed by EDC coupling with an amine PEG linker. The Fmoc functional group is removed with piperidine.

Minimum Inhibitory Concentration Assay

TABLE 3
24hr MIC of N-linked and C-linked PEGylated Amphotericin B
benchmarked against Amphotericin B.

| (PEG)ₐ | N-linked AmB | C-linked AmB | |
|---|---|---|---|
| repeat | PEGₓ CH₃ | PEGₓ CH₃ | Amphotericin B |
| units | MIC (μm) | MIC (μm) | (μm) |
| 4 | 9 | 1.18 | |
| 12 | 70 | 4 | 0.75 |
| 24 | >100 | 7 | |

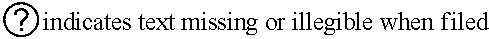

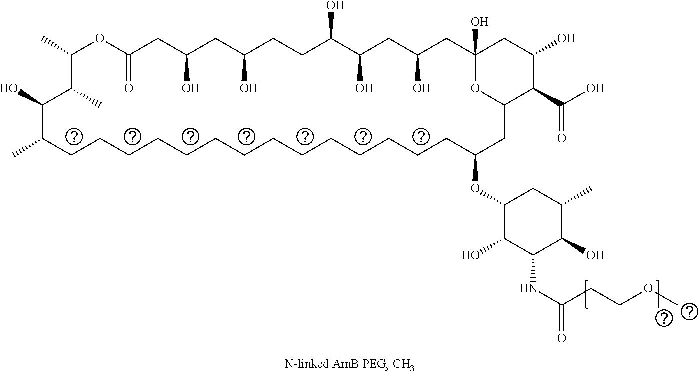

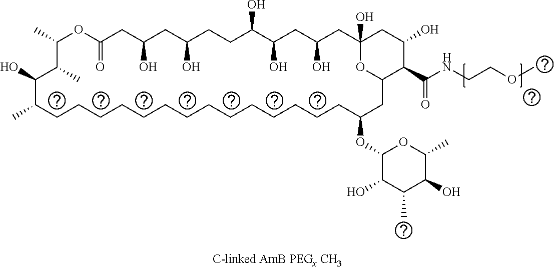

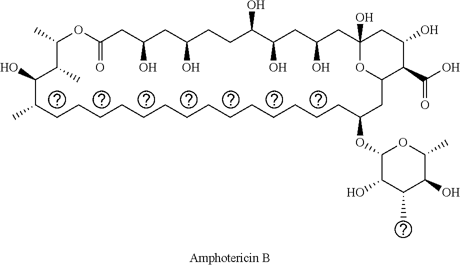 [SMILES: C[C@@H]1[C@H](O)[C@H](O[C@H]2CCCCCCCCCCCCCC[C@H](C)[C@@H](O)[C@@H](C)[C@H](C)OC(=O)C[C@H](O)C[C@H](O)CC[C@@H](O)[C@H](O)C[C@H](O)C[C@]3(O)C[C@H](O)[C@@H](C(=O)O)C(C2)O3)O[C@H](C)[C@H]1O]

The MIC assay of the N-linked and C-linked PEG conjugated Amphotericin B was conducted for 3 different PEG lengths of 4, 12 and 24 repeat units versus unconjugated Amphotericin B (Table 3). The PEG conjugated Amphotericin B and free Amphotericin B were diluted and incubated at 37° C. for 24 hrs with 10⁴1 Candida albicans cells/well according to the CLSI reference method for antifungal susceptibility testing of yeasts.

For each conjugation site, MIC increases with PEG length though the C-linked site generally has a lower MIC than the N-linked site. The C-linked Amphotericin B variants also have MIC values that are closer to the unconjugated Amphotericin B; demonstrating better activity retention.

Synthesis of Thiol Labile Carboxylic Acid Linked PEG Amphotericin B

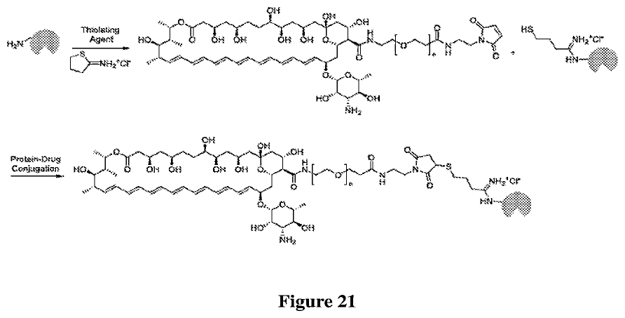

Conjugation of PEG linked Amphotericin B to hDectin1 (FIG. 21) involves a coupling reaction between nucleophilic amines or thiols with an electrophilic functionality such as NHS ester or maleimide of the PEG linker. Thiols are more reactive tan amines but naturally occurring ones are found only on free cysteines while primary amines found on lysine are much more abundant. Despite the abundance of lysine residues, they are not completely suitable because they exist in equilibrium between the protonated and free form at the physiological pH7 buffer conditions required for protein stability. This conundrum is resolved by the use of lysine residue thiolating reagents that convert amines into thiols. By adopting the thiol conjugate addition reaction with maleimide, a more hydrolysis stable linker can be used.

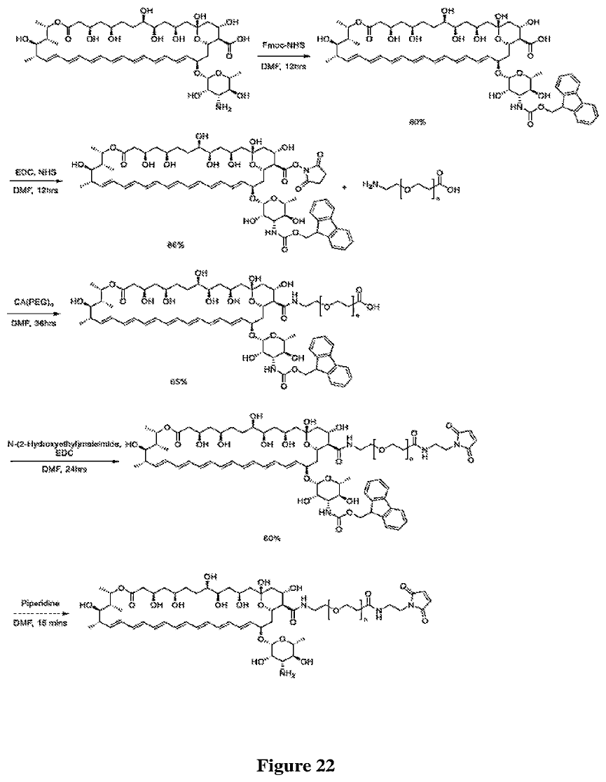

Synthesis of maleimide terminated C-linked PEG Amphotericin B (FIG. 22) begins with masking of the amino group on Amphotericin B with Fmoc. The carboxylic acid on the masked Amphotericin B was then activated to form a NHS-ester that was reacted with the carboxyl amine PEG linker. The carboxylic acid terminated PEG was in turn reacted with N-(2-hydroxyethyl)maleimide to convert the terminal end of the PEG linker into a thiol labile maleimide group. Deprotection of the Fmoc will occur only prior to conjugation with hDectin1 or hDectin1-Fc with solubilisation into aqueous medium to promote ionization of the amino group and to prevent self-reaction with other maleimide groups.

1. A soluble dimeric fusion protein comprising a first and second polypeptides, wherein the first and second polypeptides each comprises a human Dectin-1 receptor polypeptide fused to a human Fc domain via a dimerization linker, wherein the first and second polypeptides form a dimeric fusion protein via association between the dimerization linkers on each of the first and second polypeptides.

2-23. (canceled)
